(19) United States
(12) Patent Application Publication
(10) Pub. No.: US 2002/0037531 A1
(43) Pub. Date: Mar. 28, 2002

(54) Expression cloning of protein targets for phospholipids

(76) Inventors: Joan Brugge, Brookline, MA (US); Vikram Rao, Maynard, MA (US)

Correspondence Address: FOLEY, HOAG & ELIOT, LLP, PATENT GROUP, ONE POST OFFICE SQUARE, BOSTON, MA, 02109, US

(21) Appl. No.: 09/752,154

(22) Filed: Dec. 29, 2000

Related U.S. Application Data
(63) Continuation-in-part of application No. 09/735,065, filed on Dec. 11, 2000, now abandoned.
(60) Provisional application No. 60/170,009, filed on Dec. 9, 1999.

(51) Int. Cl. G01N 33/53; G01N 33/567; G01N 33/543

(52) U.S. Cl. 435/7.1; 435/7.21; 436/518

(57) ABSTRACT

One aspect of the present invention relates to methods and reagents for identifying proteins or other cellular components (collectively “LBP” or “lipid binding partner”), which bind to lipids such as phospholipids, triacylglycerides, plasmalogens or sphingolipids. In preferred embodiments, the subject method is useful for identifying LBPs that bind to phospholipids such as phosphatidylserines, phosphatidylcholines (also called lecithins), phosphatidylethanolamines, phosphatidylglycerols, phosphatidylinositols, or sphingomyelins. The LBPs can be naturally occurring, such as proteins or fragments of proteins cloned or otherwise derived from cells, or can be artificial, e.g., poypeptides which are selected from random or semi-random polypeptide libraries.

### RELATED APPLICATIONS

This application is continuation-in-part of U.S. Ser. No. 09/735,065 filed Dec. 11, 2000, which in turn claims priority to U.S. Provisional application No. 60/170,009 filed Dec. 9, 1999; the specifications of which are incorporated by reference herein.

### GOVERNMENT SUPPORT

This invention was partially funded by NIH Grant No. CA27951 and CA78773 from the National Cancer Institute; the government has certain rights to the invention.

### BACKGROUND OF THE INVENTION

It is now well recognized that dynamic changes in the phosphorylation state of intracellular phosphatidylinositol (PtdIns) play critical roles in mediating many cellular events. Phosphatidylinositol 3′-kinases (PI 3′-Ks) are a subfamily of PtdIns kinases that phosphorylate the 3′-OH (D3) position of PtdIns to create four different PtdIns derivatives: PtdIns-3-P, PtdIns-3,4-P₂, PtdIns-3,5-P₂, and PtdIns-3,4,5-P₃. Nine different isoforms of PI 3′-K have been identified in mammalian cells and they have been grouped into three classes by Domin and Waterfield based on the specific form of PI that is used as a substrate.

Singly phosphorylated PtdIns-3-P is constitutively expressed in cells and is involved in a variety of events associated with membrane protein trafficking. While all classes of PI 3′-Ks can phosphorylate PtdIns to generate this lipid, the majority of PtdIns-3-P is probably produced by Class III PI 3′-Ks which is specific for PtdIns. PtdIns-3,4-P₂ and PtdIns-3,4,5-P₃ are generated following stimulation by a wide variety of extracellular stimuli through many diverse classes of receptors. Class I PI 3′Ks phosphorylate PtdIns-4,5-P₂ to generate PtdIns-3,4,5-P₃, which can be dephosphorylated to PtdIns-3,4-P₂ by the 5′ lipid phosphatase SHIP. Alternate pathways to PtdIns-3,4-P₂ have also been described involving phosphorylation of the 4-position of PtdIns-3-P by Class II PI 3′K or an unidentified PtdIns-3-P 4-kinase, but the extent to which these enzymes contribute to PI-3,4-P₂ synthesis in not clear.

Class I PI 3′-Ks play critical roles in many essential cellular processes. Perhaps most importantly, these kinases regulate cell survival. Inhibition of class I PI 3′-Ks leads to an induction of programmed cell death or apoptosis and constitutive unregulated activation of these enzymes or downstream targets of PtdIns-3,4-P₂ and PtdIns-3,4,5-P₃ can rescue cells from cell death induced by serum deprivation, loss of matrix attachment, myc expression, and other apoptotic stimuli. These kinases also control the activation of many intracellular signaling pathways that regulate cell proliferation including Erk/MAPKs, protein translation factors (e.g. eIF-4E), and cyclins /cyclin-dependent kinases. Also, membrane trafficking events regulated by 3′PPIs control receptor internalization. In addition, PI 3′-Ks are necessary for glucose transporter recruitment to the plasma membrane and regulation of glycogen synthase kinase 3 and phosphofructokinase, indicating that 3′PPIs are critically involved in insulin-mediated events associated with glucose metabolism. Integrin affinity modulation is also blocked by pharmacological inhibitors of PI 3′-K implicating these kinases in critical events associated with leukocyte trafficking and inflammatory responses. PI 3′-K also plays an important role in regulating cell movement and cytoskeletal rearrangements. For example, 3 ′PPIs are necessary for controlling receptor-induced changes in actin assembly, the formation of lamellipodial protrusions, and cell migration through the small GTP binding protein Rac.

Because of the central importance of PI 3′-Ks in controlling cell proliferation, survival, and motility it is likely that class I PI 3′-Ks and 3′PPI binding proteins play an important role in the pathogenesis of cancer. Overexpression of PI 3′-K in chicken cells is sufficient to induce cellular transformation both in vitro and in vivo. PI 3′-K has also been implicated in the induction of Chronic Myelogenous Leukemia (CML) and Acute Lymphocytic Leukemia (ALL) by the BCR-ABL oncogene. As might be expected from its importance in cellular motility, PI 3′-K has been shown to play a role in tumor invasion and metastasis in several model systems. Recently, a role for PI 3′-K in human carcinogenesis was demonstrated by the evidence that the tumor suppressor PTEN, is a lipid phosphatase which is specific for 3′phosphate of the inositol head-group of and that elimination of the lipid phosphatase activity correlates with the oncogenic potential of PTEN mutants found in human cancers 3′PPIs.

There are several identified protein motifs that bind to 3′PPIs: PH domains, FYVE domains, SH2 domains, and C2 domains. The primary function of these domains, each of which is approximately 90 to 120 amino acids in size, is believed to be localization of the protein to high local concentrations of 3′PPIs found near active signaling complexes at the cell membrane. However, there is evidence indicating that these domains can regulate protein function as well.

The most diverse and best-characterized 3′PPI binding domains are PH domains which comprise a large family of binding modules that are known to bind proteins as well as a wide range of lipids. A subset of PH domains binds with a high affinity to PtdIns-3,4-P₂ and PtdIns-3,4,5-P₃. These PH domains are critical for the function of several signaling proteins including the serine/threonine kinases Akt and PDK1, the tyrosine kinase Btk, and the ARF-GEF Grp1.

FYVE domains are recently characterized domains that contain a zinc finger, associate exclusively with PtdIns-3-P, and are important for vesicle sorting. SH2 domains bind primarily to phosphorylated tyrosines; however, the SH2 domains of PLCγ, the Src tyrosine kinase, and the p85 subunit of PI 3′-K can also bind to PtdIns-3,4,5-P₃ with micromolar affinity. C2 domains bind to PtdIns-4,5-P₂, PtdIns-3,4-P₂, and PtdIns-3,4,5-P₃ and the specificity of lipid binding depends upon the local concentration of calcium. C2 domains are found in PKCs, PLA, and in vesicle sorting proteins such as synaptotagmin.

### SUMMARY OF THE INVENTION

The phosphatidylinositol 3-kinase (PI 3′-K) family of lipid kinases play a critical role in cell proliferation, survival, vesicle trafficking, motility, cytoskeletal rearrangements, and oncogenesis. To identify downstream effectors of PI 3′-K, we developed a novel screen to isolate proteins which bind to the major products of PI 3′-K ñ phosphatidylinositol-3,4-bisphosphate (PtdIns-3,4-P₂) and PtdIns-3,4,5-P₃. This screen uses synthetic analogs of these lipids in conjunction with libraries of proteins that are produced by coupled in vitro transcription/translation reactions. The feasibility of the screen was initially demonstrated using avidin-coated beads pre-bound to biotinylated PtdIns-3,4-P₂ and PtdIns-3,4,5-P₃ to specifically isolate the PH domain of the serine/threonine kinase Akt. We then demonstrated the utility of this technique in isolating novel 3′phosphorylated phosphatidylinositol (3′PPI) binding proteins through the preliminary screening of in vitro transcribed/translated cDNAs from a small pool expression library derived from mouse spleen. Three proteins were isolated that bound specifically to 3′PPIs. Two of these proteins have been previously characterized as PIP3BP/p42^{IP4} and the PtdIns-3,4,5-P₃-dependent serine/threonine kinase PDK1. The third protein is a novel protein that contains an SH2 domain and a PH domain which has a higher specificity for both PtdIns-3,4,5-P₃ and PtdIns-3,4-P₂ than for PtdIns-4,5-P₂. Transcripts of this novel gene (called PHISH for 3′ Phosphoinositide Interacting SH2-Containing protein) are present in every tissue analyzed but are most prominently expressed in spleen.

This invention demonstrates the utility of this technique for isolating and characterizing 3′PPI binding proteins and specifically contemplates broad applicability for the isolation of binding domains for other lipid products.

One aspect of the invention provides a method for identifying a cellular component which binds to a lipid moiety comprising:

a. providing a lipid bait moiety being derivatized to a solid support;

b. contacting the lipid bait moiety with a library of cellular components;

c. identifying those members of the cellular component library which specifically bind to the lipid bait moiety.

In preferred embodiments, the lipid bait moiety is a phospholipid, e.g. selected from the group consisting of phosphatidylethanolamines, phosphatidylcholines, phosphatidylserines, phosphatidylglycerols, phosphatidylinositols, polyphosphatidylinositols, and diphosphatidylglycerols. In certain preferred embodiments, the phospholipid is derivatized to the solid support through a cross-linking moiety which is covalently attached to a phosphate head group of the phospholipid.

In other preferred embodiments, the lipid bait moiety a plasmalogen or a sphingolipid.

In certain preferred embodiments, the library of cellular components is a polypeptide library, e.g., including at least 10 different polypeptides, more preferably at least 100, 1000, or even 10,000 different proteins. For instance, the polypeptide library can be an expression library, such as derived from replicable genetic display packages. In other embodiments, the polypeptide library is a cell lysate or partially purified protein preparation.

The identity of those members of the cellular component library which specifically bind to the lipid bait moiety can be determined by mass spectroscopy.

Another aspect of the present invention provides a screening assay comprising:

a. providing a reaction mixture including a cellular component identified as described above for its ability to specifically bind to the lipid bait moiety;

b. contacting the cellular component with a test compound;

c. determining if the test compound binds to the cellular component.

In preferred embodiments, the assay is repeated for a variegated library of at least 100 different test compounds, even more preferably at least 100, 1000 or even 10,000 different test compounds. Exemplary compounds which can be screened for activity in the subject assays include peptides, nucleic acids, carbohydrates, small organic molecules, and natural product extract libraries, such as isolated from animals, plants, fungus and/or microbes.

In certain preferred embodiments, the reaction mixture is a whole cell. In other embodiments, the reaction mixture is a cell lysate or purified protein composition.

In certain embodiments, a test compound which is identified as able to bind to the cellular component is further tested for the ability to inhibit or mimic the activity of a lipid moiety.

Still another aspect of the present invention provides a method of conducting a drug discovery business comprising:

a. providing a lipid bait moiety being derivatized to a solid support;

b. contacting the lipid bait moiety with a library of cellular components;

c. identifying those members of the cellular component library which specifically bind to the lipid bait moiety.

d. providing a reaction mixture including a cellular component identified in step (c) as able to specifically bind to the lipid bait moiety;

e. contacting the cellular component with a test compound;

f. determining if the test compound binds to the cellular component;

g. further testing those test compound identified in step (f) as able to bind to the cellular component for the ability to inhibit or mimic the activity of a lipid moiety; and

h. formulating a pharmaceutical preparation including one or more compounds identified in step (g) as able to inhibit or mimic the activity of a lipid moiety.

Yet another aspect of the invention provides a method of conducting a target discovery business comprising:

a. providing a lipid bait moiety being derivatized to a solid support;

b. contacting the lipid bait moiety with a library of cellular components;

c. identifying those members of the cellular component library which specifically bind to the lipid bait moiety.

d. licensing, to a third party, the rights for drug development for a cellular component identified in step (c) as able to specifically bind to the lipid bait moiety.

### BRIEF DESCRIPTION OF THE DRAWINGS

FIG. 1. Synthesis of biotinylated PIPₙ probes and structure of dioctanoyl derivatives

FIG. 2. The PH domain of Akt binds specifically to avidin beads pre-bound with biotinylated 3′PPIs. A. ³⁵S-labeled maltose binding protein (MBP) or MBP fused to the PH domain of Akt (MBP-PH) (lanes 1 and 5) were incubated with avidin beads alone (lanes 2 and 6), avidin beads pre-bound with PtdIns-3,4-P₂-biotin (lanes 3 and 7), avidin beads pre-bound with PtdIns-3,4,5-P₃ -biotin (lanes 4 and 8). Proteins were labeled with ³⁵S-methionine by in vitro transcription/translation of 0.5 μg of the respective genes in pCS2(+) and the binding reactions were done as described in “Experimental Procedures”. Truncated species of the MBP and MBP-PH (ΔMBP, ΔMBP-PH) result from initiation of translation at start sites after the initial AUG codon. B. Avidin beads pre-bound with PtdIns-3,4-P₂-biotin can specifically isolate MBP-PH from a pool of other proteins. 10 ng of MBP-PH DNA and/or 1 μg of DNA from a random pool from the CDNA library were transcribed/translated in the presence ³⁵S-methionine and binding reactions were performed as described in “Experimental Procedures”. Total labeled proteins (lanes 1, 4, and 7), labeled proteins bound to avidin beads (lanes 2, 5, and 8), labeled proteins bound to avidin beads pre-bound with PtdIns-3,4-P₂-biotin (lanes 3, 6, and 9). C. PtdIns-3,4,5-P₃ and PtdIns-3,4-P₂ preferentially displace MBP-PH from PtdIns-3,4,5-P₃-biotin. ³⁵S-labeled MBP-PH was bound to avidin beads coated with PtdIns-3,4,5-P₃-biotin in the presence of the indicated concentrations of PtdIns-4,5-P₂ (squares), PtdIns-3,4-P₂ (circles), PtdIns-3,4,5-P₃ (triangles) and processed as described in Experimental Proceduresî. Points represent the mean of two independent experiments.

FIG. 3. Isolation of murine isoforms of PDK1 and PIP3BP/p42^{IP4} from expression library via avidin beads pre-bound with biotinylated 3′PPIs. A. PDK1. Top panel. Specific binding of PDK1 in total pool and as single clone. Total ³⁵S-labeled proteins (lanes 1 and 5), labeled proteins bound to avidin beads (lanes 2 and 6), labeled proteins bound to avidin beads pre-bound with PtdIns-3,4-P₂-biotin (lanes 3 and 7), labeled proteins bound to avidin beads pre-bound with PtdIns-3,4,5-P₃ -biotin (lanes 4 and 8). Bottom panel. Lower diagrams show the protein domain structure of PDK1 and corresponding cDNA fragment isolated from the expression library. Nucleotide positions of putative stop and start codons (AUG) are indicated. B. PIP3BP/p42^{IP4}. Top panel. Specific binding of PIP3BP/p42^{IP4} in total pool and as single clone. Total ³⁵S-labeled proteins (lanes 1 and 5), labeled proteins bound to avidin beads (lanes 2 and 6), labeled proteins bound to avidin beads pre-bound with PtdIns-3,4-P₂-biotin (lanes 3 and 7), labeled proteins bound to avidin beads pre-bound with PtdIns-3,4,5-P₃ -biotin (lanes 4 and 8). Bottom panel. Lower diagrams show the protein domain structure of PIP3BP/p42^{IP4} and corresponding cDNA fragment isolated from the expression library. Nucleotide positions of putative stop and start codons (AUG) are indicated.

FIG. 4. Isolation of PHISH from expression library via avidin beads pre-bound with biotinylated 3′PPIs. A. Specific binding of PHISH in total pool and as single clone. Total ³⁵S-labeled proteins (lanes 1 and 5), labeled proteins bound to avidin beads (lanes 2 and 6), labeled proteins bound to avidin beads pre-bound with PtdIns-3,4-P₂-biotin (lanes 3 and 7), labeled proteins bound to avidin beads pre-bound with PtdIns-3,4,5-P₃ -biotin (lanes 4 and 8). B. PtdIns-3,4,5-P₃ and PtdIns-3,4-P₂ preferentially displace PHISH from PtdIns-3,4,5-P₃ -biotin bound to avidin beads. ³⁵S-labeled PHISH was bound to avidin beads coated with PtdIns-3,4,5-P₃-biotin in the presence of the indicated concentrations of PtdIns-4,5-P₂ (squares), PtdIns-3,4-P₂ (circles), PtdIns-3,4,5-P₃ (triangles) and processed as described in Experimental Proceduresî. Points represent the mean of two independent experiments.

FIG. 5. Nucleotide and amino acid sequence of murine PHISH. Sequence of PHISH isolated via expression cloning. Putative SH2 domain is outlined in red, predicted tyrosine phosphorylation site is outlined in green, putative PH domain is outlined in blue.

FIG. 6. Northern blot of mRNA from various murine tissues hybridized to probes derived from PHISH and GAPDH. Murine tissue RNA (10 μg per lane) was subjected to agarose gel electrophoresis, transferred to nylon membrane, and hybridized to ³²P-labeled probes derived from the coding regions of PHISH or GAPDH as described in experimental Proceduresî. Lanes: 1-brain, 2-heart, 3-lung, 4-lymph node, 5-spleen, 6-thymus. Arrows represent the mobility of the major (large arrow) and minor (small arrow) products of an in vitro transcription reaction of the PHISH gene isolated from the expression library.

### DETAILED DESCRIPTION OF THE INVENTION

A) Overview

One aspect of the present invention relates to methods and reagents for identifying proteins or other cellular components (collectively “LBP” or “lipid binding partner”), which bind to lipids such as phospholipids, triacylglycerides, plasmalogens or sphingolipids. In preferred embodiments, the subject method is useful for identifying LBPs that bind to phospholipids such as phosphatidylserines, phosphatidylcholines (also called lecithins), phosphatidylethanolamines, phosphatidylglycerols, phosphatidylinositols, or sphingomyelins. The LBPs can be naturally occurring, such as proteins or fragments of proteins cloned or otherwise derived from cells, or can be artificial, e.g., polypeptides which are selected from random or semi-random polypeptide libraries.

In general, the method of the present invention comprises providing a lipid which includes an “sequestration tag”, and contacting the lipid with a structurally diverse (variegated) library of polypeptides or other molecules under conditions wherein binding of lipids to library molecules can occur such the resulting complexes are enriched for library molecules which specifically (as opposed to non-specifically) bind the lipids. Library molecules which specifically bind to the lipids are isolated from the library, or their identity is otherwise determined, e.g., by the presence of a tag associated with the LBP which is a unique identifier of the LBP. The polypeptide library can be provided as part of a replicable genetic display package, an expression library (especially an intracellular expression library), a synthetic polypeptide library or other form.

In other embodiments, the system can be reversed and a polypeptide can be used to screen a library of structurally diverse lipids to identify lipids which selectively bind to the polypeptide.

Another aspect of the present invention relates to the LBPs which are identified by the subject method. Such molecules can be used as drug screening targets, e.g., for drugs which alter the activity of the LBP (such as its ability to bind a lipid) or which alter the level of the LBP in the cell. Moreover, the level of an LBP in a cell can be determined for diagnostic or prognostic purposes.

Where the LBP is a protein, the invention also relates to nucleic acids which encode the protein or a fragment thereof. The invention also contemplates nucleic acids which hybridize to the coding sequence for an LBP, e.g., which may be useful as amplimers, probes, primers or antisense.

Another aspect of the present invention relates to antibodies, e.g., monocolonal, purified and/or recombinant, which are immunoselective for an LBP.

Still another aspect of the present invention relates to drug screening assays for identifying compounds, e.g., such as small organic molecules (MW<1000 amu) which inhibit or potentiate the activity of an LBP. For instance, the assay can be used to identify compounds which inhibit or potentiate an intrinsic enzymatic activity of an LBP, or the ability of the LBP to bind to other molecules, e.g., to lipids, to proteins, to nucleic acids.

Yet another aspect of the present invention relates to the use the LBPs, or compounds which agonize or antagonize, as the case may be, the activity of an LBP, for the treatment or prevention of a disorder or unwanted effect mediated by a lipid.

B) Definitions

Before further description of the invention, certain terms employed in the specification, examples and appended claims are, for convenience, collected here.

“Fatty acids” are long-chain hydrocarbon molecules containing a carboxylic acid moiety at one end. The numbering of carbons in fatty acids begins with the carbon of the carboxylate group. Fatty acids that contain no carbon-carbon double bonds are termed saturated fatty acids; those that contain double bonds are unsaturated fatty acids. The numeric designations used for fatty acids come from the number of carbon atoms, followed by the number of sites of unsaturation (eg, palmitic acid is a 16-carbon fatty acid with no unsaturation and is designated by 16:0). The site of unsaturation in a fatty acid is indicated by the symbol Δ and the number of the first carbon of the double bond (e.g. palmitoleic acid is a 16-carbon fatty acid with one site of unsaturation between carbons 9 and 10, and is designated by 16:1^{Δ9}).

“Triacylglycerides” are composed of a glycerol backbone to which 3 fatty acids are esterified.

The basic structure of “phospolipids” is very similar to that of the triacylglycerides except that C-3 (sn3)of the glycerol backbone is esterified to phosphoric acid. The building block of the phospholipids is phosphatidic acid which results when the X substitution in the basic structure shown in the Figure below is a hydrogen atom. Substitutions include ethanolamine (phosphatidylethanolamine), choline (phosphatidylcholine, also called lecithins), serine (phosphatidylserine), glycerol (phosphatidylglycerol), myo-inositol (phosphatidylinositol, these compounds can have a variety in the numbers of inositol alcohols that are phosphorylated generating polyphosphatidylinositols), and phosphatidylglycerol (diphosphatidylglycerol more commonly known as cardiolipins).

“Plasmalogens” are complex membrane lipids that resemble phospholipids, principally phosphatidylcholine. The major difference is that the fatty acid at C-1 (sn1) of glycerol contains either an O-alkyl or O-alkenyl ether species. A basic O-alkenyl ether species is shown in the Figure below. One of the most potent biological molecules is platelet activating factor (PAF) which is a choline plasmalogen in which the C-2 (sn2) position of glycerol is esterified with an acetyl group instead of a long chain fatty acid.

“Sphingolipids” are composed of a backbone of sphingosine which is derived itself from glycerol. Sphingosine is N-acetylated by a variety of fatty acids generating a family of molecules referred to as ceramides. Sphingolipids predominate in the myelin sheath of nerve fibers. Sphingomyelin is an abundant sphingolipid generated by transfer of the phosphocholine moiety of phosphatidylcholine to a ceramide, thus sphingomyelin is a unique form of a phospholipid. The other major class of sphingolipids (besides the sphingomyelins) are the glycosphingolipids generated by substitution of carbohydrates to the sn1 carbon of the glycerol backbone of a ceramide. There are 4 major classes of glycosphingolipids:

Cerebrosides: contain a single moiety, principally galactose.

Sulfatides: sulfuric acid esters of galactocerebrosides.

Globosides: contain 2 or more sugars.

Gangliosides: similar to globosides except also contain sialic acid.

The term “simultaneously expressing” refers to the expression of a representative population of a polypeptide library, e.g., at least 50 percent, more preferably 75, 80, 85, 90, 95 or 98 percent of all the different polypeptide sequences of a library.

The term “random polypeptide library” refers to a set of random or semi-random polypeptides.

The language “replicable genetic display package” or “display package” describes a biological particle which has genetic information providing the particle with the ability to replicate. The package can display a fusion protein including a polypeptide derived from the variegated polypeptide library. The test polypeptide portion of the fusion protein is presented by the display package in a context which permits the polypeptide to bind to a lipid that is contacted with the display package. The display package will generally be derived from a system that allows the sampling of very large variegated polypeptide libraries. The display package can be, for example, derived from vegetative bacterial cells, bacterial spores, and bacterial viruses.

The language “differential binding means”, as well as “affinity selection” and “affinity enrichment”, refer to the separation of members of the polypeptide display library based on the differing abilities of polypeptides on the surface of each of the display packages of the library to bind to the lipid lipid. The differential binding of a lipid by test polypeptides of the display can be used in the affinity separation of those polypeptides which specifically bind the lipid from those which do not. For example, the affinity selection protocol can also include a pre- or post-enrichment step wherein display packages capable of binding “background lipids”, e.g., as a negative selection, are removed from the library. Examples of affinity selection means include affinity chromatography, immunoprecipitation, fluorescence activated cell sorting, agglutination, and plaque lifts. As described below, the affinity chromatography includes bio-panning techniques using either purified, immobilized lipid proteins or the like, as well as whole cells.

The phrases “individually selective manner” and “individually selective binding”, with respect to binding of a test polypeptide with a lipid, refers to the binding of a polypeptide to a certain protein lipid which binding is specific for, and dependent on, the molecular identity of the protein lipid.

The term “solid support” refers to a material having a rigid or semi-rigid surface. Such materials will preferably take the form of small beads, pellets, disks, chips, dishes, multi-well plates, wafers or the like, although other forms may be used. In some embodiments, at least one surface of the substrate will be substantially flat. The term “surface” refers to any generally two-dimensional structure on a solid substrate and may have steps, ridges, kinks, terraces, and the like without ceasing to be a surface.

In an exemplary embodiment of the present invention, the display package is a phage particle which comprises a polypeptide fusion coat protein that includes the amino acid sequence of a test polypeptide. Thus, a library of replicable phage vectors, especially phagemids (as defined herein), encoding a library of polypeptide fusion coat proteins is generated and used to transform suitable host cells. Phage particles formed from the chimeric protein can be separated by affinity selection based on the ability of the polypeptide associated with a particular phage particle to specifically bind a lipid. In a preferred embodiment, each individual phage particle of the library includes a copy of the corresponding phagemid encoding the polypeptide fusion coat protein displayed on the surface of that package. Exemplary phage for generating the present variegated polypeptide libraries include M13, f1, fd, If1, Ike, Xf, Pf1, Pf3, λ, T4, T7, P2, P4, φX-174, MS2 and f2.

The language “fusion protein” and “chimeric protein” are art-recognized terms which are used interchangeably herein, and include contiguous polypeptides comprising a first polypeptide covalently linked via an amide bond to one or more amino acid sequences which define polypeptide domains that are foreign to and not substantially homologous with any domain of the first polypeptide. One portion of the fusion protein comprises a test polypeptide, e.g., which can be random or semi-random. A second polypeptide portion of the fusion protein is typically derived from an outer surface protein or display anchor protein which directs the “display package” (as hereafter defined) to associate the test polypeptide with its outer surface. As described below, where the display package is a phage, this anchor protein can be derived from a surface protein native to the genetic package, such as a viral coat protein. Where the fusion protein comprises a viral coat protein and a test polypeptide, it will be referred to as a “polypeptide fusion coat protein”. The fusion protein further comprises a signal sequence, which is a short length of amino acid sequence at the amino terminal end of the fusion protein, that directs at least the portion of the fusion protein including the test polypeptide to be secreted from the cytosol of a cell and localized on the extracellular side of the cell membrane.

Gene constructs encoding fusion proteins are likewise referred to a “chimeric genes” or “fusion genes”.

The term “vector” refers to a DNA molecule, capable of replication in a host cell, into which a gene can be inserted to construct a recombinant DNA molecule.

The terms “phage vector” and “phagemid” are art-recognized and generally refer to a vector derived by modification of a phage genome, containing an origin of replication for a bacteriophage, and preferably, though optional, an origin (ori) for a bacterial plasmid. The use of phage vectors rather than the phage genome itself provides greater flexibility to vary the ratio of chimeric polypeptide/coat protein to wild-type coat protein, as well as supplement the phage genes with additional genes encoding other heterologous polypeptides, such as “auxiliary polypeptides” which may be useful in the “dual” polypeptide display constructs described below.

The language “helper phage” describes a phage which is used to infect cells containing a defective phage genome or phage vector and which functions to complement the defect. The defect can be one which results from removal or inactivation of phage genomic sequence required for production of phage particles. Examples of helper phage are M13K07.

As used herein, a “reporter gene construct” is a nucleic acid that includes a “reporter gene” operatively linked to at least one transcriptional regulatory sequence. Transcription of the reporter gene is controlled by these sequences to which they are linked.

The term “sequester”, as used herein, means to separate, segregate, remove, or bind a lipid complex, e.g., on a solid support. In preferred embodiments, a lipid complex is sequestered by a solid support such that other non-sequestered LBPs can be removed, e.g., by washing or other purification techniques. A lipid complex is “reversibly sequestered” if the process of sequestering the complex on a solid support can be reversed to yield a free complex or free LBP, e.g., in solution in a reaction mixture. In preferred embodiments, the process of sequestering a complex, or of reversing the sequestration, or both, occurs under mild conditions and in high yield, e.g., greater than at least about 40% yield.

The term “polymeric support”, as used herein, refers to a soluble or insoluble polymer to which a lipid can be covalently bonded (e.g., by through an ester functionality) by reaction with a functional group of the polymeric support. Many suitable polymeric supports are known, and include soluble polymers such as polyethylene glycols or polyvinyl alcohols, as well as insoluble polymers such as polystyrene resins. A suitable polymeric support includes functional groups such as those described below. A polymeric support is termed “soluble” if a polymer, or a polymer-supported compound, is soluble under the conditions employed. However, in general, a soluble polymer can be rendered insoluble under defined conditions. Accordingly, a polymeric support can be soluble under certain conditions and insoluble under other conditions. A polymeric support is termed “insoluble” if reaction of a lipid with the polymeric support results in an insoluble polymer-supported lipid under the conditions employed.

Abbreviations used herein include: ARF—ADP ribosylation factor; Btk—Brutons tyrosine kinase; DTT—dithiothreitol; Erk/MAPK—extracellular regulated kinase/mitogen activated protein kinase; EST—expressed sequence tag; GAP=GTPase activating protein; GAPDH=glyceraldehyde 3-phosphate dehydrogenase; GTP—guanosine triphosphate; HEPES—(N-[2-hydroxyethyl]piperazine-N′-[2-ethanesulfonic acid); kb=kilobase; SDS—sodium dodecyl sulfate; MBP=maltose binding protein; MBP-PH=maltose binding protein Akt PH domain fusion protein; NP-40—nonylphenylpolyethylene glycol; 3′PPI—3′ phosphorylated phosphatidylinositols; PAGE—polyacrylamide gel electrophoresis; PCR—polymerase chain reaction; PDK1—phosphoinositide dependent kinase 1; PH=pleckstrin homology; PI3′-K=phosphatidylinositol 3′-kinase; Pkc—protein kinase C; PLA—phopholipase A; PLCγ—phospholipase Cγ; PtdIns—phosphatidylinositol; PtdIns-3-P—phosphatidylinositol-3-monophosphate; PtdIns-3,4-P₂—phosphatidylinositol-3,4-bisphosphate; PtdIns-4,5-P₂—phosphatidylinositol-4,5-bisphosphate; PtdIns-3,4,5-P₃—phosphatidylinositol-3,4,5-trisphosphate; SDS—sodium dodecyl sulfate; SH2=Src homology 2.

C) Exemplary Embodiments of Phospholipid Baits

As set forth above, in certain embodiments, the subject method can be practiced by utilizing immobilized lipid moieties, such as phospholipids, as the bait for identifying polypeptides and other molecules capable of interacting with, and forming complexes with the lipid moiety. In certain embodiments, the subject lipid moiety is a phospolipids, such as selected from the group consisting of phosphatidylethanolamines, phosphatidylcholines, phosphatidylserines, phosphatidylglycerols, phosphatidylinositols, polyphosphatidylinositols, and diphosphatidylglycerols. Exemplary polyphosphatidylinositols include:

di C16, L-a-D-myo-Phosphatidylinositol3-monophosphate

di C8, L-a-D-myo-Phosphatidylinositol3-monophosphate

di C16, L-a-D-myo-Phosphatidylinositol3,4-diphosphate

di C8, L-a-D-myo-Phosphatidylinositol3,4-diphosphate

di C16, L-a-D-myo-Phosphatidylinositol 3,4,5-diphosphate

di C8, L-a-D-myo-Phosphatidylinositol 3,4,5-diphosphate

di C16, L-a-D-myo-Phosphatidylinositol 3,5-diphosphate

di C8, L-a-D-myo-Phosphatidylinositol 3,5-diphosphate

di C16, L-a-D-myo-Phosphatidylinositol 4-monophosphate

di C8, L-a-D-myo-Phosphatidylinositol 4-monophosphate

di C16, L-a-D-myo-Phosphatidylinositol 4,5-diphosphate

di C8, L-a-D-myo-Phosphatidylinositol 4,5-diphosphate

di C16, L-a-D-myo-Phosphatidylinositol 5-monophosphate

di C8, L-a-D-myo-Phosphatidylinositol 5-monophosphate

In other embodiments, the subject lipid moiety is a plasmalogen. In still other embodiments, the subject lipid moiety is a sphingolipid, such as may be selected from the group consisting of cerebrosides, sulfatides, globosides, and gangliosides.

In certain preferred embodiments, the subject lipid can be immobilized or incorporated into a polymer or other insoluble matrix by, for example, derivativation with one or more of subject lipid moieties derivatized to a solid support, such as glass, silicon, or a polymeric support. The support can be, inter alia, a bead, a chip, a hydrogel, etc.

In certain preferred embodiments, the subject lipid moieties are derivatized by covalent or non-covalent coupling through one or more of its fatty acid side chains, e.g., in order to present at least a portion of its head group. For example, the present invention specifically contemplates phosphatidylinositol derivatives represented by the general formula:

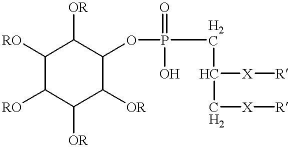

wherein

X, independently for each occurrence, represents O or S;

R, independently for each occurrence, represents hydrogen or -PO₃;

R′ represents, for each occurrence, —CH═CHR₃—L or —COR₄—L;

R₃ and R4, independently for each occurrence, represent a C6-C24 alkyl group, e.g., which may be saturated or unsaturated, branched or linear, substituted or unsubstituted; and

L represents a linker, or a linker covalently or non-covalently attached to a solid support. In certain preferred embodiments, X represents O; and L is a linker of 150-1500 amu, such a biotin.

In certain embodiments, particularly where more than one type of lipid-moiety is used as a bait (e.g., a library of different lipid moieties), a spatial array of lipid baits can be generated, e.g., for library versus library screening. For example, libraries of at least 10 different lipid moieties can be tested as baits, and more preferably libraries of at least 100 or even 1000 different lipid moieties.

The lipid moiety can be derivatived to the support by any of a number of means. In the case of phospholipids, the derivatization is preferably through a phosphate head group. As described in the appended examples, biotinylation of the phosphate head group can be used to derivatize the lipid moiety to an avidin-displaying support.

There are a large number of other chemical cross-linking agents which could be used in the present invention are known in the art. For the present invention, the preferred cross-linking agents are heterobifunctional cross-linkers, which can be used to link the lipid bait and solid support in a stepwise manner. Heterobifunctional cross-linkers provide the ability to design more specific coupling methods for conjugating the subject moieties, thereby reducing the occurrences of unwanted side reactions such as homo-lipid polymers. A wide variety of heterobifunctional cross-linkers are known in the art. These include: succinimidyl 4-(N-maleimidomethyl) cyclohexane-1-carboxylate (SMCC), m-Maleimidobenzoyl-N-hydroxysuccinimide ester (MBS); N-succinimidyl (4-iodoacetyl) aminobenzoate (SIAB), succinimidyl 4-(p-maleimidophenyl) butyrate (SMPB), 1-ethyl-3-(3-dimethylaminopropyl)carbodiimide hydrochloride (EDC); 4-succinimidyloxycarbonyl-a-methyl-a-(2-pyridyldithio)-tolune (SMPT), N-succinimidyl 3-(2-pyridyldithio)propionate (SPDP), succinimidyl 6->3-(2-pyridyldithio)propionate!hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo.

In addition to the heterobifunctional cross-linkers, there exists a number of other useful cross-linking agents including homobifunctional and photoreactive cross-linkers. Disuccinimidyl suberate (DSS), bismaleimidohexane (BMH) and dimethylpimelimidate-2 HCl (DMP) are examples of useful homobifunctional cross-linking agents, and bis-β-(4-azidosalicylamido)ethyldisulfide (BASED) and N-succinimidyl-6(4′-azido-2′-nitrophenylamino)hexanoate (SANPAH) are examples of useful photoreactive cross-linkers for use in this invention. For a review of coupling techniques which may be applied to the subject lipid moieties, see Means et al. (1990) Bioconjugate Chemistry 1:2-12.

The third component of the heterobifunctional cross-linker is the spacer arm or bridge. The bridge is the structure that connects the two reactive ends. The most apparent attribute of the bridge is its effect on steric hindrance. In some instances, a longer bridge can more easily span the distance necessary to link two complex biomolecules. For instance, SMPB has a span of 14.5 angstroms.

D) Exemplary Embodiments of Polypeptide Libraries

One goal of the present method is to identify proteins which are bound by the lipid bait. Accordingly, the present invention contemplates that any of a number of methods for trapping protein complexes using non-protein baits can be used. For instance, the proteins which are bound to the lipid bait can be identified by sequencing using mass spectroscopy. This technique can be advantageous when the source of test proteins is a cell lysate. In other embodiments, the polypeptides are associated with a tag(s) which identifies the sequence of the protein, or with the gene which encodes the protein. In still other instance, the proteins are provided as part of a spatial array for which the coordinates on the array provides the identity of the protein.

In certain preferred embodiments, the polypeptide library is provided as an expression library. For instance, a library of test polypeptides is expressed by a population of display packages to form a peptide display library. With respect to the display package on which the variegated peptide library is manifest, it will be appreciated from the discussion provided herein that the display package will preferably be able to be (i) genetically altered to encode heterologous peptide, (ii) maintained and amplified in culture, (iii) manipulated to display the peptide-containing gene product in a manner permitting the peptide to interact with a lipid during an affinity separation step, and (iv) affinity separated while retaining the nucleotide sequence encoding the test polypeptide (herein “peptide gene”) such that the sequence of the peptide gene can be obtained. In preferred embodiments, the display remains viable after affinity separation.

Ideally, the display package comprises a system that allows the sampling of very large variegated peptide display libraries, rapid sorting after each affinity separation round, and easy isolation of the peptide gene from purified display packages or further manipulation of that sequence in the secretion mode. The most attractive candidates for this type of screening are prokaryotic organisms and viruses, as they can be amplified quickly, they are relatively easy to manipulate, and large number of clones can be created. Preferred display packages include, for example, vegetative bacterial cells, bacterial spores, and most preferably, bacterial viruses (especially DNA viruses). However, the present invention also contemplates the use of eukaryotic cells, including yeast and their spores, as potential display packages.

In addition to commercially available kits for generating phage display libraries (e.g. the Pharmacia Recombinant Phage Antibody System, catalog no. 27-9400-01; and the Stratagene SurfZAP™ phage display kit, catalog no. 240612), examples of methods and reagents particularly amenable for use in generating the variegated peptide display library of the present invention can be found in, for example, the Ladner et al. U.S. Pat. No. 5,223,409; the Kang et al. International Publication No. WO 92/18619; the Dower et al. International Publication No. WO 91/17271; the Winter et al. International Publication WO 92/20791; the Markland et al. International Publication No. WO 92/15679; the Breitling et al. International Publication WO 93/01288; the McCafferty et al. International Publication No. WO 92/01047; the Garrard et al. International Publication No. WO 92/09690; the Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982. These systems can, with modifications described herein, be adapted for use in the subject method.

When the display is based on a bacterial cell, or a phage which is assembled periplasmically, the display means of the package will comprise at least two components. The first component is a secretion signal which directs the recombinant peptide to be localized on the extracellular side of the cell membrane (of the host cell when the display package is a phage). This secretion signal can be selected so as to be cleaved off by a signal peptidase to yield a processed, “mature” peptide. The second component is a display anchor protein which directs the display package to associate the test polypeptide with its outer surface. As described below, this anchor protein can be derived from a surface or coat protein native to the genetic package.

When the display package is a bacterial spore, or a phage whose protein coating is assembled intracellularly, a secretion signal directing the peptide to the inner membrane of the host cell is unnecessary. In these cases, the means for arraying the variegated peptide library comprises a derivative of a spore or phage coat protein amenable for use as a fusion protein.

In some instances it may be necessary to introduce an unstructured polypeptide linker region between portions of the chimeric protein, e.g., between the test polypeptide and display polypeptide. This linker can facilitate enhanced flexibility of the chimeric protein allowing the test polypeptide to freely interact with a lipid by reducing steric hindrance between the two fragments, as well as allowing appropriate folding of each portion to occur. The linker can be of natural origin, such as a sequence determined to exist in random coil between two domains of a protein. Alternatively, the linker can be of synthetic origin. For instance, the sequence (Gly₄Ser)₃ can be used as a synthetic unstructured linker. Linkers of this type are described in Huston et al. (1988) PNAS 85:4879; and U.S. Pat. Nos. 5,091,513 and 5,258,498. Naturally occurring unstructured linkers of human origin are preferred as they reduce the risk of immunogenicity.

In the instance wherein the display package is a phage, the cloning site for the test polypeptide gene sequences in the phagemid should be placed so that it does not substantially interfere with normal phage function. One such locus is the intergenic region as described by Zinder and Boeke, (1982) Gene 19: 1-10.

The number of possible combinations in a peptide library can get large as the length is increased and selection criteria for degenerating at each position is relaxed. To sample as many combinations as possible depends, in part, on the ability to recover large numbers of transformants. For phage with plasmid-like forms (as filamentous phage), electrotransformation provides an efficiency comparable to that of phage-transfection with in vitro packaging, in addition to a very high capacity for DNA input. This allows large amounts of vector DNA to be used to obtain very large numbers of transformants. The method described by Dower et al. (1988) Nucleic Acids Res., 16:6127-6145, for example, may be used to transform fd-tet derived recombinants at the rate of about 10⁷ transformants/ug of ligated vector into E. coli (such as strain MCl1061), and libraries may be constructed in fd-tet B1 of up to about 3 ×10⁸ members or more. Increasing DNA input and making modifications to the cloning protocol within the ability of the skilled artisan may produce increases of greater than about 10-fold in the recovery of transformants, providing libraries of up to 10¹⁰ or more recombinants.

As will be apparent to those skilled in the art, in embodiments wherein high affinity peptides are sought, an important criteria for the present selection method can be that it is able to discriminate between peptides of different affinity for a particular lipid, and preferentially enrich for the peptides of highest affinity. Applying the well known principles of peptide affinity and valence (i.e. avidity), it is understood that manipulating the display package to be rendered effectively monovalent can allow affinity enrichment to be carried out for generally higher binding affinities (i.e. binding constants in the range of 10⁶ to 10¹⁰ M⁻¹) as compared to the broader range of affinities isolable using a multivalent display package. To generate the monovalent display, the natural (i.e. wild-type) form of the surface or coat protein used to anchor the peptide to the display can be added at a high enough level that it almost entirely eliminates inclusion of the peptide fusion protein in the display package. Thus, a vast majority of the display packages can be generated to include no more than one copy of the peptide fusion protein (see, for example, Garrad et al. (1991) Bio/Technology 9:1373-1377). In a preferred embodiment of a monovalent display library, the library of display packages will comprise no more than 5 to 10% polyvalent displays, and more preferably no more than 2% of the display will be polyvalent, and most preferably, no more than 1% polyvalent display packages in the population. The source of the wild-type anchor protein can be, for example, provided by a copy of the wild-type gene present on the same construct as the peptide fusion protein, or provided by a separate construct altogether. However, it will be equally clear that by similar manipulation, polyvalent displays can be generated to isolate a broader range of binding affinities. Such peptides can be useful, for example, in purification protocols where avidity can be desirable.

i) Phages as Display Packages

Bacteriophage are attractive prokaryotic-related organisms for use in the subject method. Bacteriophage are excellent candidates for providing a display system of the variegated polypeptide library as there is little or no enzymatic activity associated with intact mature phage, and because their genes are inactive outside a bacterial host, rendering the mature phage particles metabolically inert. In general, the phage surface is a relatively simple structure. Phage can be grown easily in large numbers, they are amenable to the practical handling involved in many potential mass screening programs, and they carry genetic information for their own synthesis within a small, simple package. As the polypeptide gene is inserted into the phage genome, choosing the appropriate phage to be employed in the subject method will generally depend most on whether (i) the genome of the phage allows introduction of the polypeptide gene either by tolerating additional genetic material or by having replaceable genetic material; (ii) the virion is capable of packaging the genome after accepting the insertion or substitution of genetic material; and (iii) the display of the polypeptide on the phage surface does not disrupt virion structure sufficiently to interfere with phage propagation.

One concern presented with the use of phage is that the morphogenetic pathway of the phage determines the environment in which the polypeptide will have opportunity to fold. Periplasmically assembled phage are preferred as the displayed polypeptides may contain essential disulfides, and such polypeptides may not fold correctly within a cell. However, in certain embodiments in which the display package forms intracellularly (e.g., where λ phage are used), it has been demonstrated in other instances that disulfide-containing polypeptides can assume proper folding after the phage is released from the cell.

Another concern related to the use of phage, but also pertinent to the use of bacterial cells and spores as well, is that multiple infections could generate hybrid displays that carry the gene for one particular test polypeptide yet have two or more different test polypeptides on their surfaces. Therefore, it can be preferable, though optional, to minimize this possibility by infecting cells with phage under conditions resulting in a low multiple-infection.

For a given bacteriophage, the preferred display means is a protein that is present on the phage surface (e.g. a coat protein). Filamentous phage can be described by a helical lattice; isometric phage, by an icosahedral lattice. Each monomer of each major coat protein sits on a lattice point and makes defined interactions with each of its neighbors. Proteins that fit into the lattice by making some, but not all, of the normal lattice contacts are likely to destabilize the virion by aborting formation of the virion as well as by leaving gaps in the virion so that the nucleic acid is not protected. Thus in bacteriophage, unlike the cases of bacteria and spores, it is generally important to retain in the polypeptide fusion proteins those residues of the coat protein that interact with other proteins in the virion. For example, when using the M13 cpVIII protein, the entire mature protein will generally be retained with the polypeptide fragment being added to the N-terminus of cpVIII, while on the other hand it can suffice to retain only the last 100 carboxy terminal residues (or even fewer) of the M13 cpIII coat protein in the polypeptide fusion protein.

Under the appropriate induction, the test polypeptide library is expressed and exported, as part of the fusion protein, to the bacterial cytoplasm, such as when the λ phage is employed. The induction of the fusion protein(s) may be delayed until some replication of the phage genome, synthesis of some of the phage structural-proteins, and assembly of some phage particles has occurred. The assembled protein chains then interact with the phage particles via the binding of the anchor protein on the outer surface of the phage particle. The cells are lysed and the phage bearing the library-encoded test polypeptides (that corresponds to the specific library sequences carried in the DNA of that phage) are released and isolated from the bacterial debris.

To enrich for and isolate phage which encodes a selected test polypeptide, and thus to ultimately isolate the nucleic acid sequences (the polypeptide gene) themselves, phage harvested from the bacterial debris are affinity purified. As described below, when a test polypeptide which specifically binds a particular lipid is desired, the lipid can be used to retrieve phage displaying the desired test polypeptide. The phage so obtained may then be amplified by infecting into host cells. Additional rounds of affinity enrichment followed by amplification may be employed until the desired level of enrichment is reached.

The enriched polypeptide-phage can also be screened with additional detection-techniques such as expression plaque (or colony) lift (see, e.g., Young and Davis, Science (1983) 222:778-782) whereby a labeled lipid is used as a probe.

a) Filamentous Phage

Filamentous bacteriophages, which include M13, f1, fd, If1, Ike, Xf, Pf1, and Pf3, are a group of related viruses that infect bacteria. They are termed filamentous because they are long, thin particles comprised of an elongated capsule that envelopes the deoxyribonucleic acid (DNA) that forms the bacteriophage genome. The F pili filamentous bacteriophage (Ff phage) infect only gram-negative bacteria by specifically adsorbing to the tip of F pili, and include fd, f1 and M13.

Compared to other bacteriophage, filamentous phage in general are attractive and M13 in particular is especially attractive because: (i) the 3-D structure of the virion is known; (ii) the processing of the coat protein is well understood; (iii) the genome is expandable; (iv) the genome is small; (v) the sequence of the genome is known; (vi) the virion is physically resistant to shear, heat, cold, urea, guanidinium chloride, low pH, and high salt; (vii) the phage is a sequencing vector so that sequencing is especially easy; (viii) antibiotic-resistance genes have been cloned into the genome with predictable results (Hines et al. (1980) Gene 11:207-218); (ix) it is easily cultured and stored, with no unusual or expensive media requirements for the infected cells, (x) it has a high burst size, each infected cell yielding 100 to 1000 M13 progeny after infection; and (xi) it is easily harvested and concentrated (Salivar et al. (1964) Virology 24: 359-371). The entire life cycle of the filamentous phage M13, a common cloning and sequencing vector, is well understood. The genetic structure of M13 is well known, including the complete sequence (Schaller et al. in The Single-Stranded DNA Phages eds. Denhardt et al. (NY: CSHL Press, 1978)), the identity and function of the ten genes, and the order of transcription and location of the promoters, as well as the physical structure of the virion (Smith et al. (1985) Science 228:1315-1317; Raschad et al. (1986) Microbiol Dev 50:401-427; Kuhn et al. (1987) Science 238:1413-1415; Zimmerman et al. (1982) J Biol Chem 257:6529-6536; and Banner et al. (1981) Nature 289:814-816). Because the genome is small (6423 bp), cassette mutagenesis is practical on RF M13 (Current Protocols in Molecular Biology, eds. Ausubel et al. (NY: John Wiley & Sons, 1991)), as is single-stranded oligonucleotide directed mutagenesis (Fritz et al. in DNA Cloning, ed by Glover (Oxford, UK: IRC Press, 1985)). M13 is a plasmid and transformation system in itself, and an ideal sequencing vector. M13 can be grown on Rec-strains of E. coli. The M13 genome is expandable (Messing et al. in The Single-Stranded DNA Phages, eds Denhardt et al. (NY: CSHL Press, 1978) pages 449-453; and Fritz et al., supra) and M13 does not lyse cells. Extra genes can be inserted into M13 and will be maintained in the viral genome in a stable manner.

The mature capsule or Ff phage is comprised of a coat of five phage-encoded gene products: cpVIII, the major coat protein product of gene VIII that forms the bulk of the capsule; and four minor coat proteins, cpIII and cpIV at one end of the capsule and cpVII and cpIX at the other end of the capsule. The length of the capsule is formed by 2500 to 3000 copies of cpVIII in an ordered helix array that forms the characteristic filament structure. The gene III-encoded protein (cpIII) is typically present in 4 to 6 copies at one end of the capsule and serves as the receptor for binding of the phage to its bacterial host in the initial phase of infection. For detailed reviews of Ff phage structure, see Rasched et al., Microbiol. Rev., 50:401-427 (1986); and Model et al.,in The Bacteriophages, Volume 2, R. Calendar, Ed., Plenum Press, pp. 375-456 (1988).

The phage particle assembly involves extrusion of the viral genome through the host cell's membrane. Prior to extrusion, the major coat protein cpVIII and the minor coat protein cpIII are synthesized and transported to the host cell's membrane. Both cpVIII and cpIII are anchored in the host cell membrane prior to their incorporation into the mature particle. In addition, the viral genome is produced and coated with cpV protein. During the extrusion process, cpV-coated genomic DNA is stripped of the cpV coat and simultaneously recoated with the mature coat proteins.

Both cpIII and cpVIII proteins include two domains that provide signals for assembly of the mature phage particle. The first domain is a secretion signal that directs the newly synthesized protein to the host cell membrane. The secretion signal is located at the amino terminus of the polypeptide and lipids the polypeptide at least to the cell membrane. The second domain is a membrane anchor domain that provides signals for association with the host cell membrane and for association with the phage particle during assembly. This second signal for both cpVIII and cpIII comprises at least a hydrophobic region for spanning the membrane.

The 50 amino acid mature gene VIII coat protein (cpVIII) is synthesized as a 73 amino acid precoat (Ito et al. (1979) PNAS 76:1199-1203). cpVIII has been extensively studied as a model membrane protein because it can integrate into lipid bilayers such as the cell membrane in an asymmetric orientation with the acidic amino terminus toward the outside and the basic carboxy terminus toward the inside of the membrane. The first 23 amino acids constitute a typical signal-sequence which causes the nascent polypeptide to be inserted into the inner cell membrane. An E. coli signal peptidase (SP-I) recognizes amino acids 18, 21, and 23, and, to a lesser extent, residue 22, and cuts between residues 23 and 24 of the precoat (Kuhn et al. (1985) J. Biol. Chem. 260:15914-15918; and Kuhn et al. (1985) J. Biol. Chem. 260:15907-15913). After removal of the signal sequence, the amino terminus of the mature coat is located on the periplasmic side of the inner membrane; the carboxy terminus is on the cytoplasmic side. About 3000 copies of the mature coat protein associate side-by-side in the inner membrane.

The sequence of gene VIII is known, and the amino acid sequence can be encoded on a synthetic gene. Mature gene VIII protein makes up the sheath around the circular ssDNA. The gene VIII protein can be a suitable anchor protein because its location and orientation in the virion are known (Banner et al. (1981) Nature 289:814-816). Preferably, the polypeptide is attached to the amino terminus of the mature M13 coat protein to generate the phage display library. As set out above, manipulation of the concentration of both the wild-type cpVIII and Ab/cpVIII fusion in an infected cell can be utilized to decrease the avidity of the display and thereby enhance the detection of high affinity polypeptides directed to the lipid(s).

Another vehicle for displaying the polypeptide is by expressing it as a domain of a chimeric gene containing part or all of gene III, e.g., encoding cpIII. When monovalent displays are required, expressing the polypeptide as a fusion protein with cpIII can be a preferred embodiment, as manipulation of the ratio of wild-type cpIII to chimeric cpIII during formation of the phage particles can be readily controlled. This gene encodes one of the minor coat proteins of M13. Genes VI, VII, and IX also encode minor coat proteins. Each of these minor proteins is present in about 5 copies per virion and is related to morphogenesis or infection. In contrast, the major coat protein is present in more than 2500 copies per virion. The gene VI, VII, and IX proteins are present at the ends of the virion; these three proteins are not posttranslationally processed (Rasched et al. (1986) Ann Rev. Microbiol. 41:507-541). In particular, the single-stranded circular phage DNA associates with about five copies of the gene III protein and is then extruded through the patch of membrane-associated coat protein in such a way that the DNA is encased in a helical sheath of protein (Webster et al. in The Single-Stranded DNA Phages, eds Dressler et al. (NY:CSHL Press, 1978).

Manipulation of the sequence of cpIII has demonstrated that the C-terminal 23 amino acid residue stretch of hydrophobic amino acids normally responsible for a membrane anchor function can be altered in a variety of ways and retain the capacity to associate with membranes. Ff phage-based expression vectors were first described in which the cpIII amino acid residue sequence was modified by insertion of heterologous polypeptide (Parmely et al., Gene (1988) 73:305-318; and Cwirla et al., PNAS (1990) 87:6378-6382) or an amino acid residue sequence defining a single chain polypeptide domain (McCafferty et al., Science (1990) 348:552-554). It has been demonstrated that insertions into gene III can result in the production of novel protein domains on the virion outer surface. (Smith (1985) Science 228:1315-1317; and de la Cruz et al. (1988) J. Biol. Chem. 263:4318-4322). The polypeptide gene maybe fused to gene III at the site used by Smith and by de la Cruz et al., at a codon corresponding to another domain boundary or to a surface loop of the protein, or to the amino terminus of the mature protein.

Generally, the successful cloning strategy utilizing a phage coat protein, such as cpIII of filamentous phage fd, will provide expression of a polypeptide chain fused to the N-terminus of a coat protein (e.g., cpIII) and transport to the inner membrane of the host where the hydrophobic domain in the C-terminal region of the coat protein anchors the fusion protein in the membrane, with the N-terminus containing the polypeptide chain protruding into the periplasmic space.

Similar constructions could be made with other filamentous phage. Pf3 is a well known filamentous phage that infects Pseudomonos aerugenosa cells that harbor an IncP-I plasmid. The entire genome has been sequenced ((Luiten et al. (1985) J. Virol. 56:268-276) and the genetic signals involved in replication and assembly are known (Luiten et al. (1987) DNA 6:129-137). The major coat protein of PF3 is unusual in having no signal peptide to direct its secretion. The sequence has charged residues ASP-7, ARG-37, LYS-40, and PHE44 which is consistent with the amino terminus being exposed. Thus, to cause a polypeptide to appear on the surface of Pf3, a tripartite gene can be constructed which comprises a signal sequence known to cause secretion in P. aerugenosa, fused in-frame to a gene fragment encoding the polypeptide sequence, which is fused in-frame to DNA encoding the mature Pf3 coat protein. Optionally, DNA encoding a flexible linker of one to 10 amino acids is introduced between the polypeptide gene fragment and the Pf3 coat-protein gene. This tripartite gene is introduced into Pf3 so that it does not interfere with expression of any Pf3 genes. Once the signal sequence is cleaved off, the polypeptide is in the periplasm and the mature coat protein acts as an anchor and phage-assembly signal.

b) Bacteriophage φX174

The bacteriophage φX174 is a very small icosahedral virus which has been thoroughly studied by genetics, biochemistry, and electron microscopy (see The Single Stranded DNA Phages (eds. Den hardt et al. (NY:CSHL Press, 1978)). Three gene products of φX174 are present on the outside of the mature virion: F (capsid), G (major spike protein, 60 copies per virion), and H (minor spike protein, 12 copies per virion). The G protein comprises 175 amino acids, while H comprises 328 amino acids. The F protein interacts with the single-stranded DNA of the virus. The proteins F, G, and H are translated from a single mRNA in the viral infected cells. As the virus is so tightly constrained because several of its genes overlap, φX174 is not typically used as a cloning vector due to the fact that it can accept very little additional DNA. However, mutations in the viral G gene (encoding the G protein) can be rescued by a copy of the wild-type G gene carried on a plasmid that is expressed in the same host cell (Chambers et al. (1982) Nuc Acid Res 10:6465-6473). In one embodiment, one or more stop codons are introduced into the G gene so that no G protein is produced from the viral genome. The variegated polypeptide gene library can then be fused with the nucleic acid sequence of the H gene. An amount of the viral G gene equal to the size of polypeptide gene fragment is eliminated from the φX174 genome, such that the size of the genome is ultimately unchanged. Thus, in host cells also transformed with a second plasmid expressing the wild-type G protein, the production of viral particles from the mutant virus is rescued by the exogenous G protein source. Where it is desirable that only one test polypeptide be displayed per φX174 particle, the second plasmid can further include one or more copies of the wild-type H protein gene so that a mix of H and test polypeptide/H proteins will be predominated by the wild-type H upon incorporation into phage particles.

c) Large DNA Phage

Phage such as λ or T4 have much larger genomes than do M13 or φX174 and have more complicated 3-D capsid structures than M13 or φX174 with more coat proteins to choose from. In embodiments of the invention whereby the test polypeptide library is processed and assembled into a functional form and associates with the bacteriophage particles within the cytoplasm of the host cell, bacteriophage λ and derivatives thereof are examples of suitable vectors. The intracellular morphogenesis of phage λ can potentially prevent protein domains that ordinarily contain disulfide bonds from folding correctly. However, variegated libraries expressing a population of functional polypeptides, which include such bonds, have been generated in λ phage. (Huse et al. (1989) Science 246:1275-1281; Mullinax et al. (1990) PNAS 87:8095-8099; and Pearson et al. (1991) PNAS 88:2432-2436). Such strategies take advantage of the rapid construction and efficient transformation abilities of λ phage.

When used for expression of polypeptide sequences (ixogenous nucleotide sequences), may be readily inserted into a λ vector. For instance, variegated polypeptide libraries can be constructed by modification of λ ZAP II through use of the multiple cloning site of a λ ZAP II vector (Huse et al. supra).

ii) Bacterial Cells as Display Packages

Recombinant polypeptides are able to cross bacterial membranes after the addition of appropriate secretion signal sequences to the N-terminus of the protein (Better et al (1988) Science 240:1041-1043; and Skerra et al. (1988) Science 240:1038-1041). In addition, recombinant polypeptides have been fused to outer membrane proteins for surface presentation. For example, one strategy for displaying polypeptides on bacterial cells comprises generating a fusion protein by inserting the polypeptide into cell surface exposed portions of an integral outer membrane protein (Fuchs et al. (1991) Bio/Technology 9:1370-1372). In selecting a bacterial cell to serve as the display package, any well-characterized bacterial strain will typically be suitable, provided the bacteria may be grown in culture, engineered to display the test polypeptide library on its surface, and is compatible with the particular affinity selection process practiced in the subject method. Among bacterial cells, the preferred display systems include Salmonella typhirnurium, Bacillus subtilis, Pseudomonas aeruginosa, Vibrio cholerae, Klebsiella pneumonia, Neisseria gonorrhoeae, Neisseria meningitidis, Bacteroides nodosus, Moraxella bovis, and especially Escherichia coli. Many bacterial cell surface proteins useful in the present invention have been characterized, and works on the localization of these proteins and the methods of determining their structure include Benz et al. (1988) Ann Rev Microbiol 42: 359-393; Balduyck et al. (1985) Biol Chem Hoppe-Seyler 366:9-14; Ehrmann et al (1990) PNAS 87:7574-7578; Heijne et al. (1990) Protein Engineering 4:109-112; Ladner et al. U.S. Pat. No. 5,223,409; Ladner et al. WO88/06630; Fuchs et al. (1991) Bio/technology 9:1370-1372; and Goward et al. (1992) TIBS 18:136-140.

To further illustrate, the LamB protein of E coli is a well understood surface protein that can be used to generate a variegated library of test polypeptides on the surface of a bacterial cell (see, for example, Ronco et al. (1990) Biochemie 72:183-189; van der Weit et al. (1990) Vaccine 8:269-277; Charabit et al. (1988) Gene 70:181-189; and Ladner U.S. Pat. No. 5,222,409). LamB of E. coli is a porin for maltose and maltodextrin transport, and serves as the receptor for adsorption of bacteriophages λ and K10. LamB is transported to the outer membrane if a functional N-terminal signal sequence is present (Benson et al. (1984) PNAS 81:3830-3834). As with other cell surface proteins, LamB is synthesized with a typical signal-sequence which is subsequently removed. Thus, the variegated polypeptide gene library can be cloned into the LamB gene such that the resulting library of fusion proteins comprise a portion of LamB sufficient to anchor the protein to the cell membrane with the test polypeptide fragment oriented on the extracellular side of the membrane. Secretion of the extracellular portion of the fusion protein can be facilitated by inclusion of the LamB signal sequence, or other suitable signal sequence, as the N-terminus of the protein.

The E. coli LamB has also been expressed in functional form in S. typhimurium (Harkki et al. (1987) Mol Gen Genet 209:607-611), V. cholerae (Harkki et al. (1986) Microb Pathol 1:283-288), and K. pneumonia (Wehmeier et al. (1989) Mol Gen Genet 215:529-536), so that one could display a population of test polypeptides in any of these species as a fusion to E. coli LamB. Moreover, K. pneumonia expresses a maltoporin similar to LamB which could also be used. In P. aeruginosa, the D1 protein (a homologue of LamB) can be used (Trias et al. (1988) Biochem Biophys Acta 938:493-496). Similarly, other bacterial surface proteins, such as PAL, OmpA, OmpC, OmpF, PhoE, pilin, BtuB, FepA, FhuA, IutA, FecA and FhuE, may be used in place of LamB as a portion of the display means in a bacterial cell.

In another exemplary embodiment, the fusion protein can be derived using the FliTrx™ Random Polypeptide Display Library (Invitrogen). That library is a diverse population of random dodecapolypeptides inserted within the thioredoxin active-site loop inside the dispensable region of the bacterial flagellin gene (fliC). The resultant recombinant fusion protein (FLITRX) is exported and assembled into partially functional flagella on the bacterial cell surface, displaying the random polypeptide library.

Polypeptides are fused in the middle of thioredoxin, therefore, both their N- and C-termini are anchored by thioredoxin's tertiary structure. This results in the display of a constrained polypeptide. By contrast, phage display proteins are fused to the N-terminus of phage coat proteins in an unconstrained manner. The unconstrained molecules possess many degrees of conformational freedom which may result in the lack of proper interaction with the lipid molecule. Without proper interaction, many potential protein-protein interactions may be missed.

Moreover, phage display is limited by the low expression levels of bacteriophage coat proteins. FliTrx™ and similar methods can overcome this limitation by using a strong promoter to drive expression of the test polypeptide fusions that are displayed as multiple copies.

According to the present invention, it is contemplated that the FliTrx vector can be modified to provide, similar to the illustrated vectors of the attached figures, a vector which is differentially spliced in mammalian cells to yield a secreted, soluble test polypeptide.

iii) Bacterial Spores as Display Packages

Bacterial spores also have desirable properties as display package candidates in the subject method. For example, spores are much more resistant than vegetative bacterial cells or phage to chemical and physical agents, and hence permit the use of a great variety of affinity selection conditions. Also, Bacillus spores neither actively metabolize nor alter the proteins on their surface. However, spores have the disadvantage that the molecular mechanisms that trigger sporulation are less well worked out than is the formation of M13 or the export of protein to the outer membrane of E. coli, though such a limitation is not a serious detractant from their use in the present invention.

Bacteria of the genus Bacillus form endospores that are extremely resistant to damage by heat, radiation, desiccation, and toxic chemicals (reviewed by Losick et al. (1986) Ann Rev Genet 20:625-669). This phenomenon is attributed to extensive intermolecular cross-linking of the coat proteins. In certain embodiments of the subject method, such as those which include relatively harsh affinity separation steps, Bacillus spores can be the preferred display package. Endospores from the genus Bacillus are more stable than are, for example, exospores from Streptomyces. Moreover, Bacillus subtilis forms spores in 4 to 6 hours, whereas Streptomyces species may require days or weeks to sporulate. In addition, genetic knowledge and manipulation is much more developed for B. subtilis than for other spore-forming bacteria.

Viable spores that differ only slightly from wild-type are produced in B. subtilis even if any one of four coat proteins is missing (Donovan et al. (1987) J Mol Biol 196:1-10). Moreover, plasmid DNA is commonly included in spores, and plasmid encoded proteins have been observed on the surface of Bacillus spores (Debro et al. (1986) J Bacteriol 165:258-268). Thus, it can be possible during sporulation to express a gene encoding a chimeric coat protein comprising a polypeptide of the variegated gene library, without interfering materially with spore formation.

To illustrate, several polypeptide components of B. subtilis spore coat (Donovan et al. (1987) J Mol Biol 196:1-10) have been characterized. The sequences of two complete coat proteins and amino-terminal fragments of two others have been determined. Fusion of the test polypeptide sequence to cotC or cotD fragments is likely to cause the polypeptide to appear on the spore surface. The genes of each of these spore coat proteins are preferred as neither cotC or cotD are post-translationally modified (see Ladner et al. U.S. Pat. No. 5,223,409).

iv) Selecting Peptides from the Display Mode

Upon expression, the variegated polypeptide display is subjected to affinity enrichment in order to select for test polypeptides which bind preselected lipids. The term “affinity separation” or “affinity enrichment” includes, but is not limited to: (1) affinity chromatography utilizing immobilized lipids, and (2) precipitation using soluble lipids. In each embodiment, the library of display packages are ultimately separated based on the ability of the associated test polypeptide to bind the lipid of interest. See, for example, the Ladner et al. U.S. Pat. No. 5,223,409; the Kang et al. International Publication No. WO 92/18619; the Dower et al. International Publication No. WO 91/17271; the Winter et al. International Publication WO 92/20791; the Markland et al. International Publication No. WO 92/15679; the Breitling et al. International Publication WO 93/01288; the McCafferty et al. International Publication No. WO 92/01047; the Garrard et al. International Publication No. WO 92/09690; and the Ladner et al. International Publication No. WO 90/02809. In most preferred embodiments, the display library will be pre-enriched for peptides specific for the lipid by first contacting the display library with any negative controls or other lipids for which differential binding by the test polypeptide is desired. Subsequently, the non-binding fraction from that pre-treatment step is contacted with the lipid and peptides from the display which are able to specifically bind the lipid are isolated.

With respect to affinity chromatography, it will be generally understood by those skilled in the art that a great number of chromatography techniques can be adapted for use in the present invention, ranging from column chromatography to batch elution, and including ELISA and biopanning techniques. Typically, where lipid is or can be immobilized on an insoluble carrier, such as sepharose or polyacrylamide beads, or, alternatively, the wells of a microtiter plate.

The population of display packages is applied to the affinity matrix under conditions compatible with the binding of the test polypeptide to the lipid. The population is then fractionated by washing with a solute that does not greatly effect specific binding of polypeptides to the lipid, but which substantially disrupts any non-specific binding of the display package to the lipid or matrix. A certain degree of control can be exerted over the binding characteristics of the polypeptides recovered from the display library by adjusting the conditions of the binding incubation and subsequent washing. The temperature, pH, ionic strength, divalent cation concentration, and the volume and duration of the washing can select for polypeptides within a particular range of affinity and specificity. Selection based on slow dissociation rate, which is usually predictive of high affinity, is a very practical route. This may be done either by continued incubation in the presence of a saturating amount of free lipid (if available), or by increasing the volume, number, and length of the washes. In each case, the rebinding of dissociated polypeptide-display package is prevented, and with increasing time, display packages of higher and higher affinity are recovered. Moreover, additional modifications of the binding and washing procedures may be applied to find polypeptides with special characteristics. The affinities of some peptides are dependent on ionic strength or cation concentration. This is a useful characteristic for peptides to be used in affinity purification of various proteins when gentle conditions for removing the protein from the peptide are required. Specific examples are polypeptides which depend on Ca⁺⁺ for lipid binding activity and which lose or gain binding affinity in the presence of EGTA or other metal chelating agent. Such peptides may be identified in the recombinant polypeptide library by a double screening technique isolating first those that bind the lipid in the presence of Ca⁺⁺, and by subsequently identifying those in this group that fail to bind in the presence of EGTA, or vice-versa.

After “washing” to remove non-specifically bound display packages, when desired, specifically bound display packages can be eluted by either specific desorption (using excess lipid) or non-specific desorption (using pH, polarity reducing agents, or chaotropic agents). In preferred embodiments, the elution protocol does not kill the organism used as the display package such that the enriched population of display packages can be further amplified by reproduction. The list of potential eluants includes salts (such as those in which one of the counter ions is Na⁺, NH₄⁺, Rb⁺, SO₄²⁻, H₂PO₄−, citrate, K⁺, Li⁺, Cs⁺, HSO₄−, CO₃²⁻, Ca²⁺, Sr²⁺, Cl⁻, PO₄²⁻, HCO₃−, Mg₂⁺, Ba₂⁺, Br⁻, HPO₄²⁻, or acetate), acid, heat, and, when available, soluble forms of the lipid. Because bacteria continue to metabolize during the affinity separation step and are generally more susceptible to damage by harsh conditions, the choice of buffer components (especially eluates) can be more restricted when the display package is a bacteria rather than for phage or spores. Neutral solutes, such as ethanol, acetone, ether, or urea, are examples of other agents useful for eluting the bound display packages.

In preferred embodiments, affinity enriched display packages are iteratively amplified and subjected to further rounds of affinity separation until enrichment of the desired binding activity is detected. In certain embodiments, the specifically bound display packages, especially bacterial cells, need not be eluted per se, but rather, the matrix bound display packages can be used directly to inoculate a suitable growth media for amplification.

Where the display package is a phage particle, the fusion protein generated with the coat protein can interfere substantially with the subsequent amplification of eluted phage particles, particularly in embodiments wherein the cpIII protein is used as the display anchor. Even though present in only one of the 5-6 tail fibers, some peptide constructs because of their size and/or sequence, may cause severe defects in the infectivity of their carrier phage. This causes a loss of phage from the population during reinfection and amplification following each cycle of panning. In one embodiment, the peptide can be derived on the surface of the display package so as to be susceptible to proteolytic cleavage which severs the covalent linkage of at least the target binding sites of the displayed peptide from the remaining package. For instance, where the cpIII coat protein of M13 is employed, such a strategy can be used to obtain infectious phage by treatment with an enzyme which cleaves between the test polypeptide portion and cpIII portion of a tail fiber fusion protein (e.g. such as the use of an enterokinase cleavage recognition sequence).

To further minimize problems associated with defective infectivity, DNA prepared from the eluted phage can be transformed into host cells by electroporation or well known chemical means. The cells are cultivated for a period of time sufficient for marker expression, and selection is applied as typically done for DNA transformation. The colonies are amplified, and phage harvested for a subsequent round(s) of panning.

After isolation of display packages which encode polypeptides having a desired binding specificity for the lipid, the test polypeptides for each of the purified display packages can be tested for biological activity in the secretion mode of the subject method.

(v) Generations of Polypeptide Libraries

The variegated polypeptide libraries of the subject method can be generated by any of a number of methods, and, though not limited by, preferably exploit recent trends in the preparation of chemical libraries. For instance, chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential test sequences. The synthesis of degenerate oligonucleotides is well known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

As used herein, “variegated” refers to the fact that a population of peptides is characterized by having a peptide sequence which differ from one member of the library to the next. For example, in a given peptide library of n amino acids in length, the total number of different peptide sequences in the library is given by the product of {V₁×V₂× . . . Vₙ₋₁×Vₙ} where each vₙ represents the number different amino acid residues occurring at position n of the peptide. In a preferred embodiment of the present invention, the peptide display collectively produces a peptide library including at least 96 to 10⁷ different peptides, so that diverse peptides may be simultaneously assayed for the ability to interact with the lipid.

In one embodiment, the test polypeptide library is derived to express a combinatorial library of peptides which are not based on any known sequence, nor derived from cDNA. That is, the sequences of the library are largely, if not entirely, random. It will be evident that the peptides of the library may range in size from dipeptides to large proteins.

In another embodiment, the peptide library is derived to express a combinatorial library of peptides which are based at least in part on a known polypeptide sequence or a portion thereof (though preferably not a cDNA library). That is, the sequences of the library is semi-random, being derived by combinatorial mutagenesis of a known sequence(s). See, for example, Ladner et al. PCT publication WO 90/02909; Garrard et al., PCT publication WO 92/09690; Marks et al. (1992) J. Biol. Chem. 267:16007-16010; Griffths et al. (1993) EMBO J 12:725-734; Clackson et al. (1991) Nature 352:624-628; and Barbas et al. (1992) PNAS 89:4457-4461. Accordingly, polypeptide(s) which are known binding partners for a lipid can be mutagenized by standard techniques to derive a variegated library of polypeptide sequences which can further be screened for agonists and/or antagonists. The purpose of screening such combinatorial peptide libraries is to generate, for example, homologs of known polypeptides which can act as either agonists or antagonists, or alternatively, possess novel activities all together. To illustrate, a ligand can be engineered by the present method to provide more efficient binding or specificity to a cognate receptor, yet still retain at least a portion of an activity associated with wild-type ligand. Thus, combinatorially-derived homologs can be generated to have an increased potency relative to a naturally occurring form of the protein. Likewise, homologs can be generated by the present approach to act as antagonists, in that they are able to mimic, for example, binding to the lipid, yet not induce any biological response, thereby inhibiting the action of authentic ligand.

In preferred embodiments, the combinatorial polypeptides are in the range of 3-100 amino acids in length, more preferably at least 5-50, and even more preferably at least 10, 13, 15, 20 or 25 amino acid residues in length. Preferably, the polypeptides of the library are of uniform length. It will be understood that the length of the combinatorial peptide does not reflect any extraneous sequences which may be present in order to facilitate expression, e.g., such as signal sequences or invariant portions of a fusion protein.

The harnessing of biological systems for the generation of polypeptide diversity is now a well established technique which can be exploited to generate the peptide libraries of the subject method. The source of diversity is the combinatorial chemical synthesis of mixtures of oligonucleotides. Oligonucleotide synthesis is a well-characterized chemistry that allows tight control of the composition of the mixtures created. Degenerate DNA sequences produced are subsequently placed into an appropriate genetic context for expression as polypeptides.

There are two principal ways in which to prepare the required degenerate mixture. In one method, the DNAs are synthesized a base at a time. When variation is desired at a base position dictated by the genetic code a suitable mixture of nucleotides is reacted with the nascent DNA, rather than the pure nucleotide reagent of conventional polynucleotide synthesis. The second method provides more exact control over the amino acid variation. First, trinucleotide reagents are prepared, each trinucleotide being a codon of one (and only one) of the amino acids to be featured in the polypeptide library. When a particular variable residue is to be synthesized, a mixture is made of the appropriate trinucleotides and reacted with the nascent DNA. Once the necessary “degenerate” DNA is complete, it must be joined with the DNA sequences necessary to assure the expression of the polypeptide, as discussed in more detail below, and the complete DNA construct must be introduced into the cell.

Whatever the method may be for generating diversity at the codon level, chemical synthesis of a degenerate gene sequence can be carried out in an automatic DNA synthesizer, and the synthetic genes can then be ligated into an appropriate gene for expression. The purpose of a degenerate set of genes is to provide, in one mixture, all of the sequences encoding the desired set of potential test polypolypeptide sequences. The synthesis of degenerate oligonucleotides is well known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

E) Exemplification

Given the diversity of cellular responses dependent on the products of PI 3′-K, it is probable that many as yet unidentified proteins bind to and are regulated by PtdIns-3,4,5-P₃ and PtdIns-3,4-P₂. Identification of these proteins and the elucidation of their role in cellular signaling will be critical to our understanding of these cellular functions as well as to diseases such as cancer. In order to isolate novel 3′PPI binding proteins we developed a screen using in vitro coupled transcription/translation technology. in conjunction with the use of synthetic biotinylated PtdIns-3,4-P₂ and PtdIns-3,4,5-P₃ ligands. We show in this report that this screen can isolate 3′PPI binding proteins with high specificity and selectivity from among a vast excess and diversity of other non-specific proteins. We report an initial demonstration of the effectiveness of the system using the PH domain of the serine/threonine kinase Akt, a known 3′PPI binder, as a model. Second, we demonstrate the utility of this technique in isolating other 3′PPI binding proteins by screening a small pool expression library derived from mouse spleen MRNA. Three 3′PPI binding proteins were identified in this initial screen. Two of these proteins have been previously characterized, PIP3BP/p42 ^{IP4}and PDK1, thereby establishing positive controls that the system operates as predicted. Importantly, the third protein is a novel protein of unknown function that contains both a PH domain and an SH2 domain.

### EXPERIMENTAL PROCEDURES

Cloning and Expression of Akt PH domain. The PH domain of Akt was cloned as a fusion protein to maltose binding protein (MBP) in the expression vector pMAL2B (New England Biolabs). The N-terminal 130 amino acids of murine Akt1 in the vector pJ3? (a gift of P. Tsichlis, Fox-Chase Cancer Center) was amplified by PCR using Pfu polymerase with the coding strand primer 5i-cgatcgggatccatggaacag-3ë (upstream of the myc tag in pJ3? and contains a BamH1 site) and the non coding strand primer 5i-ccctgaattctcactgggtga-3′ (from Akt amino acid 130 and encodes an EcoR1 site). Both maltose binding protein alone and the MBP-Akt PH domain fusion construct were subcloned into the in vitro translation vector pCS2(+) under the control of the SP6 promoter. The proteins were expressed by transcribing/translating 0.01-1 μg of DNA of MBP or MBP-Akt PH with the Promega TnT Coupled Reticulocyte Lysate System using SP6 RNA polymerase and ³⁵S-methionine (in vivo labeling grade, Amersham) according to the manufacturers protocol.

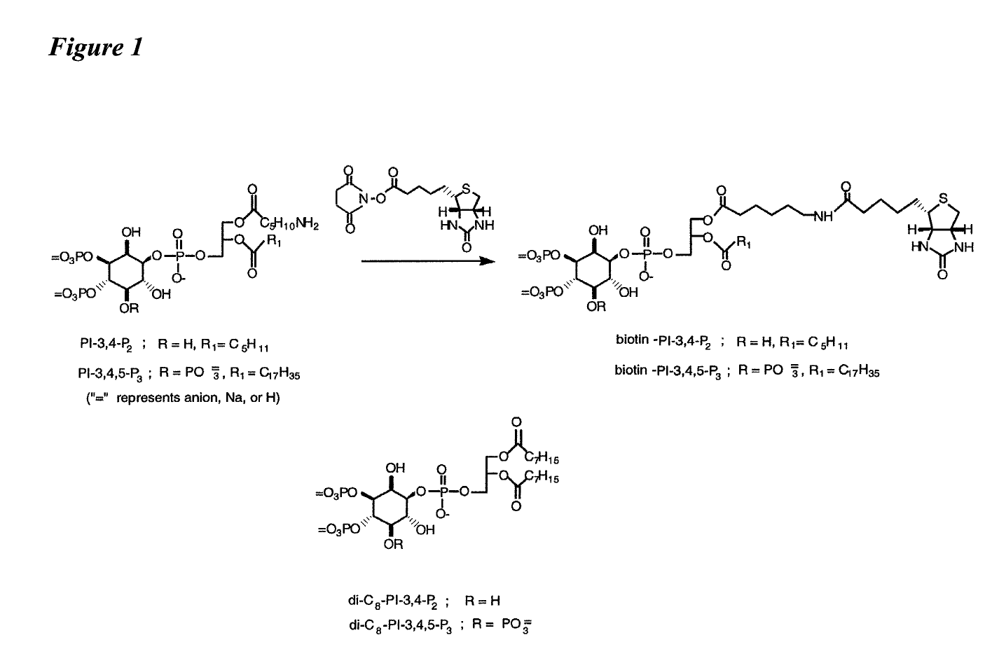

Synthesis of Biotinylated and Non-Biotinylated Phosphatidylinositols. All chemically-synthesized probes were prepared using a methyl D-glucopyranoside as the chiral starting material for the inositol head group; different phosphate substitution patterns were elaborated using the Ferrier rearrangement of suitably protected glucose-derived precursors. Di-C₈ PtdIns-3,4-P₂ and PtdIns-3,4,5-P₃ were prepared by modifications of the synthesis of the dipalmitoyl analogs. Biotinylated phosphoinositides were prepared from the sn-2-aminohexanoyl derivatives of PtdIns-3,4-P₂ and PtdIns-3,4,5-P₃ by condensation of the active ester of biotin with the water-soluble PIPₙ analog in the presence of a mild base (FIG. 1). The biotinylated probes, bPtdIns-3,4-P₂ and bPtdIns-3,4,5-P₃, were purified by ion exchange chromatography and employed as aqueous solutions for attachment to streptavidin-coated surfaces

Binding and Isolation of Radiolabeled In Vitro Translated Proteins with Biotinylated Phosphoinositides. Avidin beads (Ultralink immobilized Neutravidin™, Pierce Chemical) were washed twice with 5 volumes of wash/binding (WB) buffer (10 mM HEPES pH=7.4, 150 mM NaCl, 0.5% NP-40, 5 mM DTT). The beads were then reconstituted in 2×volume of WB buffer and the biotinylated phosphoinositide was added. Generally, 0.1 μl of 100 μM biotinylated lipid was bound per 1 μl of packed avidin beads. The biotinylated lipid was incubated with the beads for 1-2 hours at 4° C. with gentle agitation and then washed twice with 10×-bead volume of WB buffer to remove excess ligand. Control beads without biotinylated lipid were prepared in an identical manner but without the addition of the lipid. 5 μl of the ³⁵S-labelling reaction containing the in vitro transcribed/translated protein was then added to the tubes and the protein was allowed to bind for 2 hours at 4° C. with gentle agitation. The tubes were then centrifuged briefly, the beads were washed 2×with 0.5 ml WB buffer, and the bound proteins were eluted by boiling the beads in 20 μl Laemmli sample buffer containing 5% 2-mercaptoethanol. The proteins were then separated on a 13.75% SDS-PAGE gel. Following electrophoresis the gel was soaked in EnHance (Dupont/NEN) for 1 hour and then in a 7.5% w/v solution of PEG-3350 for 1 hour. The gel was then dried and subjected to autoradiography.

Competition Binding of Isolated Proteins for Non-biotinylated Lipids over Biotinylated Lipids. Competition binding experiments were performed exactly as described above for standard binding of radiolabeled proteins to biotinylated lipids except that avidin beads which had been pre-bound with the biotinylated diC₇ PtdIns-3,4,5-P₃ or PtdIns-3,4-P₂ were incubated with the in vitro translated proteins in the presence of 1 pM to 100 μM of non-biotinylated lipids (diC₈ forms of PtdIns-3,4,5-P₃, PtdIns-3,4-P₂, PtdIns-4,5-P₂ [Echelon Research Laboratories, Salt Lake City, Utah]). Binding was quantitated by measuring the intensities of bands from the autoradiogram or by scintillation counting of the eluted proteins in Laemmli sample buffer using Opti-fluor (Packard). Binding curves were modeled by the equation:

% bound=[IC₅₀/(IC₅₀+C)]×100

where % bound represents the quantity of protein bound to the biotinylated lipid coated beads in the presence of a concentration C of competing lipid. IC₅₀ is the molar excess of competitor C required to reduce the % bound to 50% of its maximal value in the absence of competitor.

In Vitro Translation Expression Cloning. A murine spleen cDNA library containing 2×10⁵ independent clones was divided into 1700 individual small pools. The cDNAs in this library had been cloned into the in vitro transcription vector pCS2(+) under control of the SP6 promoter. Individual cDNAs from positive pools were isolated from the other cDNAs of the pool using a 96-well format as described previously. The individual cDNAs were sequenced (Harvard Biopolymer Facility) and compared with known sequences by searching GenBank databases.

Northern Blotting. Northern blotting was performed as described in Current Protocols in Molecular Biology (1999). A ³²P-labeled double stranded DNA probe was made a PCR fragment from the respective gene using random primed oligonucleotide synthesis (Prime-a-Gene, Promega). Murine tissue RNA was a gift of Dr. W. Swat (Harvard Medical School).

### RESULTS AND DISCUSSION

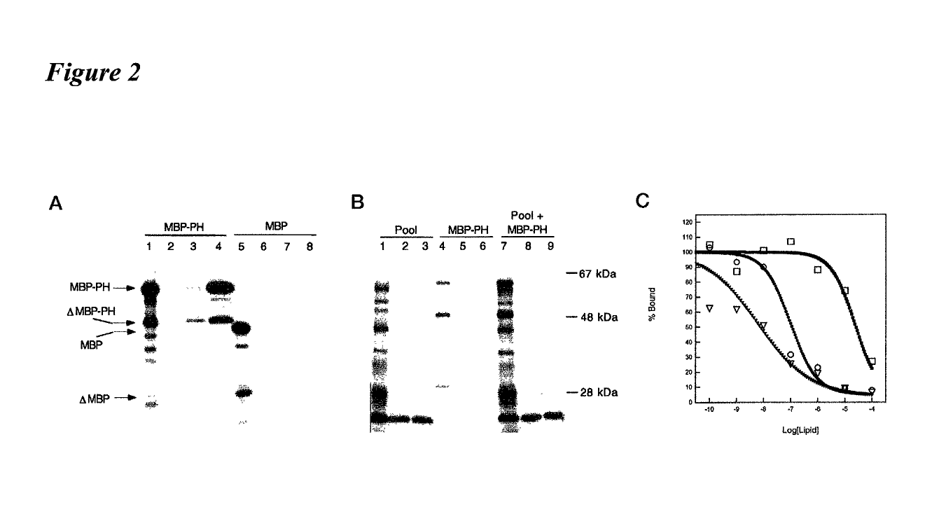

Biotinylatedforms of PtdIns-3,4-P₂ and PtdIns-3,4,5-P₃ specifically bind to the in vitro translated PH domain of Akt and can be used for affinity isolation. In order to test whether cloning of phosphoinositide binding proteins through coupled in vitro transcription/translation expression was feasible, we examined whether biotinylated forms of 3′PPIs could be used to affinity isolate a known 3′PPI binding domain that had been produced by a coupled in vitro transcription/translation system. We first pre-bound avidin-coated beads with diC₇-analogs of PtdIns-3,4,5-P₃ and PtdIns-3,4-P₂ that had been biotinylated on the ω-end of the sn-1-aminohexanoyl derivatives (see Materials and Methods and FIG. 1). The beads were then incubated with ³⁵S-methionine-labeled proteins generated by in vitro transcription/translation of cDNAs encoding either maltose binding protein (MBP) or MBP fused to the PH domain of Akt. Both genes were cloned into the in vitro transcription vector pCS2(+) under the control of the SP6 promoter. The PH domain of Akt is known to bind 3′PPIs with high affinity and served as a positive control to determine optimal binding conditions and specificity. As shown in FIG. 2A, the fusion protein of MBP-Akt PH bound to avidin beads that were pre-bound with the biotinylated forms of either PtdIns-3,4,5-P₃ or PtdIns-3,4-P₂. The MBP-PH fusion protein failed to bind to the unmodified avidin beads, and MBP failed to bind to either the avidin beads alone or to the beads pre-bound with the biotinylated phosphoinositides. These data demonstrate that the biotinylated 3′PPIs can effectively and specifically isolate an in vitro translated form of a phosphoinositide-binding domain.

The goal of this study is to isolate novel 3′PPI binding proteins from among pools of in vitro transcribed/translated proteins from a small pool expression library. In order to specifically isolate 3′PPI binding proteins from among other proteins expressed in the library it is necessary that the system used for screening have a high specificity for 3′PPI binding proteins and a low background affinity for non-specific binding proteins. The system must also be sufficiently sensitive to detect small amounts of a 3′PPI binding protein in a large background of non-specific binding proteins. To determine if our system conformed to these criteria we investigated whether biotinylated PtdIns-3,4,5-P₃ and PtdIns-3,4-P₂ could specifically isolate the MBP-Akt PH domain fusion protein after having been diluted into a pool from the in vitro translation library.

There are approximately 100 independent clones in each pool of cDNA from our pCS2mouse spleen cDNA library and the total DNA concentration was approximately 1 μg/μl for each pool. Therefore 10 ng of the plasmid encoding MBP-Akt PH fusion protein was mixed with 1 μg of DNA of a random pool from the library in order to approximate the amount of 3′PPI binding protein that would be found in a random pool under the conditions of our screen. As is shown in FIG. 2B, biotinylated PtdIns-3,4-P₂-beads captured the MBP-Akt PH fusion protein from among the other proteins in the pool. However, some proteins in the library, e.g. the 25 kDa protein, bound to the avidin beads in both the presence and absence of ligand, most likely through a non-specific hydrophobic interaction. These results provided the proof of concept that this approach is feasible for isolation of 3′PPI binding domains.

Finally we examined whether our binding conditions allowed the PH domain of Akt to distinguish between phosphoinositides phosphorylated at the 3′ position from phosphoinositides lacking a phosphate at the 3′ position. We performed competition binding experiments using unbiotinylated forms of PtdIns-3,4,5-P₃, PtdIns-3,4-P₂, and PtdIns-4,5-P₂ to compete for biotinylated PtdIns-3,4,5-P₃ binding to the MBP-Akt PH fusion protein (FIG. 2C). PtdIns-3,4,5-P₃ and PtdIns-3,4-P₂ displaced the biotinylated PtdIns-3,4,5-P₃ at concentrations of 1000 to 10,000-fold lower than PtdIns-4,5-P₂ showing that the specificity of the Akt PH domain for the 3′ phosphate is preserved under our conditions. The data also shows that PtdIns-3,4,5-P₃ binds more tightly to the Akt PH domain than does PtdIns-3,4-P₂ and this is consistent with two published studies on the affinity of the PH domain of Akt for phosphorylated phosphoinositides which rank the order of affinities as PtdIns-3,4,5-P₃>PtdIns-3,4-P₂>>PtdIns-4,5-P₂. This result demonstrates that our system preserves the specificity of protein binding to different species of phosphorylated phosphoinositides.

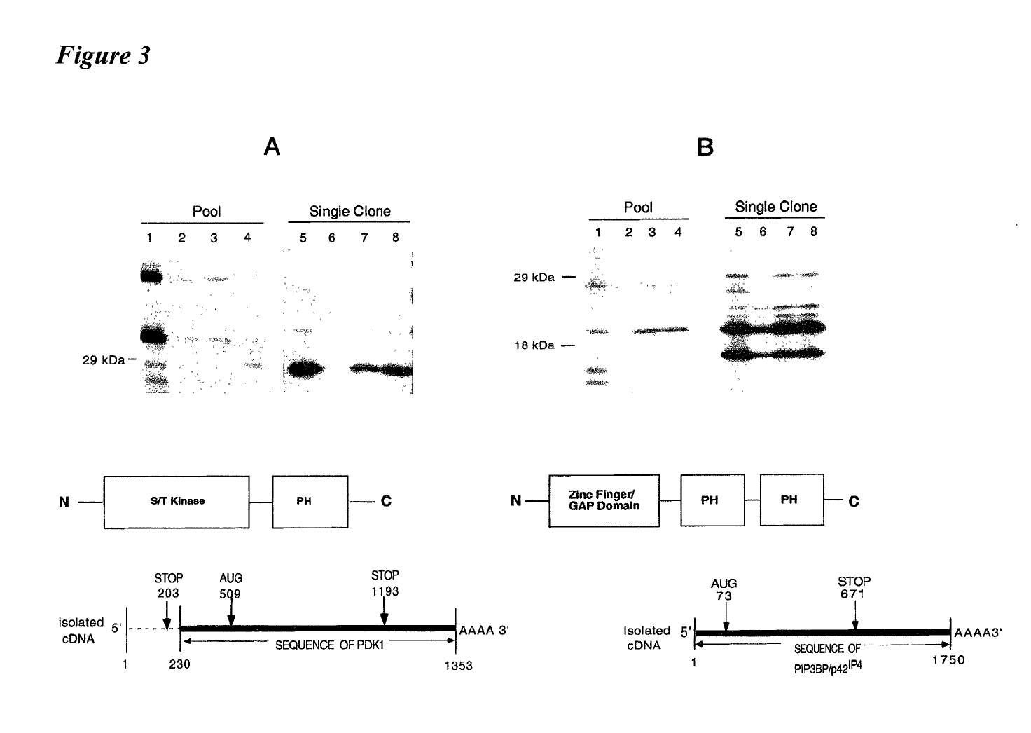

Isolation of the murine isoforms of PDK1 and PIP3BP/p42^{IP4} by small pool expression cloning. After an initial screening of 500 pools of the library using the biotinylated phosphoinositides, we isolated three 3′PPI binding proteins. We observed a protein of approximately 25 kDa in a single pool which appeared to bind exclusively to biotinylated PtdIns-3,4,5-P₃ (FIG. 3A, top panel left). When the cDNA for the protein was isolated from the other cDNAs in the pool, the expressed purified protein was also found to bind to PtdIns-3,4-P₂ but with an apparent lower affinity than for PtdIns-3,4,5-P₃ (FIG. 3A, top panel right). Sequencing of the cloned cDNA revealed that it was identical to the C-terminal 319 amino acids of murine phosphoinositide dependent kinase 1 (PDK1). PDK1 is a ubiquitously expressed 559 amino acid (65 kDa) protein which contains an N-terminal serine/threonine kinase domain and a C-terminal PH domain. In agreement with our results, the PH domain of PDK1 has recently been found to bind with a four- fold higher affinity to PtdIns-3,4,5-P₃ than to PtdIns-3,4-P₂ but with much lower affinity to PtdIns-4,5-P₂. The fragment of PDK1 that we isolated contains the entire C-terminal PH domain and approximately half of the serine/threonine kinase domain (FIG. 3A, bottom panel). However, the first 230 nucleotides of the isolated mRNA are not the sequence of PDK1 and the first in-frame AUG codon appears at the end of the kinase domain such that only the entire PH domain of PDK1 is translated. The initial 230 non-coding nucleotides could either be due to an alternative splicing of PDK1, or an artifact from the construction of the library.

Another 3′PPI binding protein identified in our screen was a protein of approximately 25 kDa that bound tightly to both biotinylated PtdIns-3,4-P₂ and PtdIns-3,4,5-P₃ but also had some residual binding to the avidin beads (FIG. 3B, top panel). Upon isolation and sequencing of the cDNA from the pool, the gene was found to encode the C-terminal 210 amino acids of a protein which has>95% amino acid homology to 42 kDa inositol-1,3,4,5-tetrakisphosphate binding proteins isolated from both porcine brain called p42^{IP4}, and bovine brain called PIP3BP. PIP3BP/p42^{IP4}is a protein of 374 amino acids which contains an N-terminal zinc-finger domain which has homology to GTPase activating proteins for the ARF family of small G-proteins, and two tandem PH domains at the C-terminal end of the protein (FIG. 3B, bottom panel). The fragment of the gene that we isolated contains the entire C-terminal PH domain and approximately half of the N-terminal PH domain but contains none of the putative ARF-GAP domain. The C-terminal PH domain contains a consensus sequence for high affinity binding of 3′PPIs. The N-terminal PH domain does not contain this sequence but two studies have shown that it displays some specificity for 3′PPIs.

The function of PIP3BP/p42^{IP4}is currently unknown although it is highly expressed in the brain and is thought to play a role in vesicle and membrane transport because of the putative ARF-GAP domain. Interestingly, a closely related yeast protein called Gcs1 has been shown to be an ARF-GAP and is important in proper cytoskeletal organization and actin polymerization in yeast. A recent study has also shown that PIP3BP is localized to the nucleus but translocates to the plasma membrane upon activation of PI 3′-K; however, the functional significance of both the nuclear localization and the PI 3′-K dependent translocation is unknown.

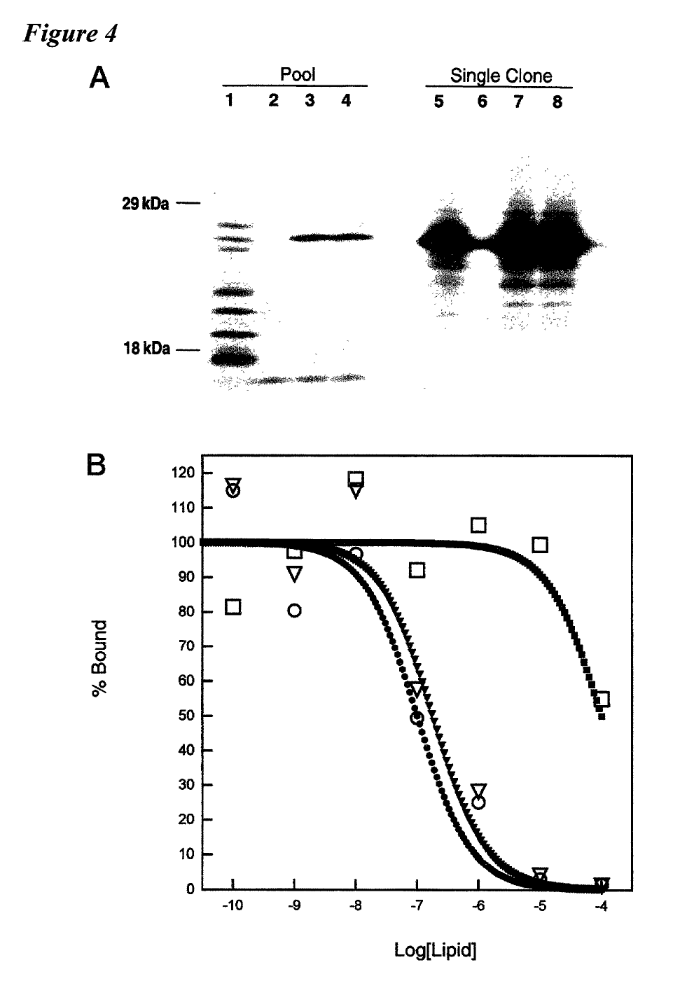

Isolation of PHISH, a Novel 3 ′PPI Binding Protein Containing both PH and SH2 Domains. We isolated a novel 3′PPI binding protein from the library that migrated at approximately 30 kDa and, similar to PIP₃BP/p42^{IP4}, bound tightly to both biotinylated PtdIns-3,4-P₂ and PtdIns-3,4,5-P₃ but not to the avidin beads alone (FIG. 4A). In competition binding studies the in vitro transcribed/translated protein was found to bind with equal affinity to both PtdIns-3,4,5-P₃ and PtdIns-3,4-P₂ but with significantly lower affinity to PtdIns-4,5-P₂, thus demonstrating the specificity of the protein for 3′PPIs (FIG. 4B).

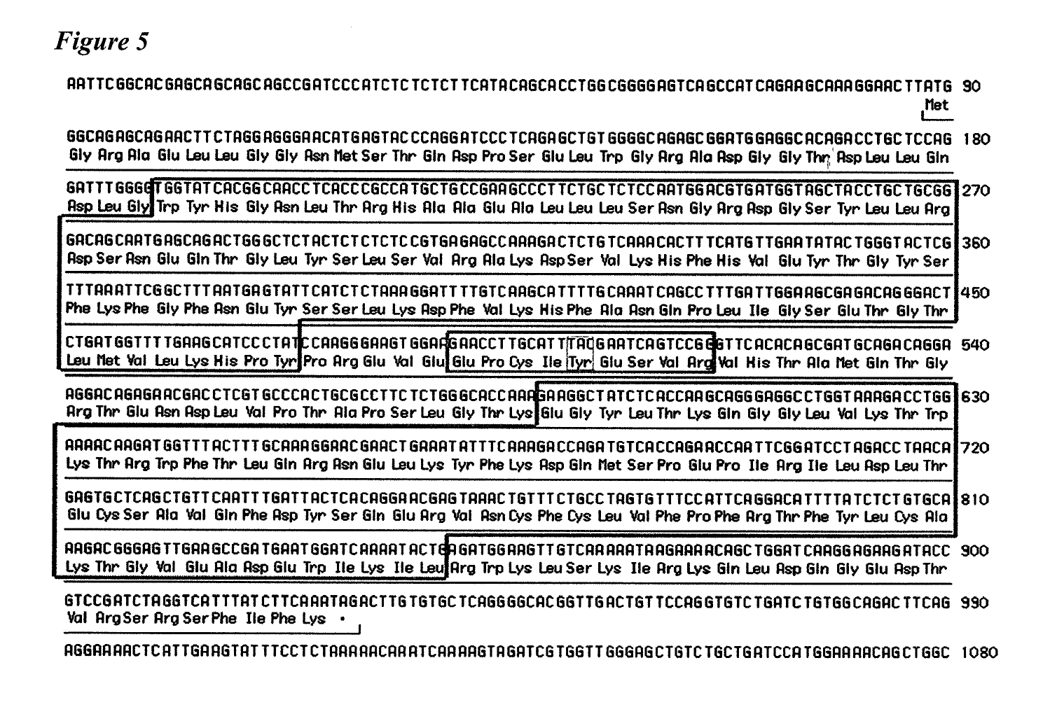

The gene fragment containing the sequence for the protein was approximately 1.3 kb in length and encoded an open reading frame from the beginning of the fragment to a stop codon at nucleotide position 927 (FIG. 5). A putative Kozak initiator (ATG) codon for methionine is present at position 87 and initiation of translation from this codon is consistent with the observed the size of the translated protein (280 amino acids and 30 kDa). However, since the sequence upstream of this ATG did not contain any in-frame stop codons it was difficult to determine if the isolated gene fragment encoded the entire coding sequence of the gene or if more coding sequences existed upstream.

The amino acid sequence contains coding regions for both an SH2 domain and a C-terminal PH domain. The SH2 domain, which encompasses nucleotides 200 to 470, is most similar to the SH2 domain of the neural adaptor protein Nck (35% identical, 59% homologous at the amino acid level). The PH domain, which encompasses nucleotides 590 to 840, is most similar to the PH domain of Akt (39% identical, 63% homologous at the amino acid level) and contains the consensus sequence for high affinity binding of 3′PPIs. There is also a tyrosine (Y139) located between the SH2 and PH domains which could be phosphorylated in stimulated cells since the sequence surrounding this tyrosine is a putative consensus motif for phosphorylation by tyrosine kinases. The existence of a putative phosphotyrosine binding SH2 domain, a 3′PPI binding PH domain, and a sequence for phosphorylation of tyrosine strongly suggest that this protein plays a role in cellular signaling. We have named this protein PHISH for í3′ Phosphoinositide Interacting SH2-Containing proteiní.

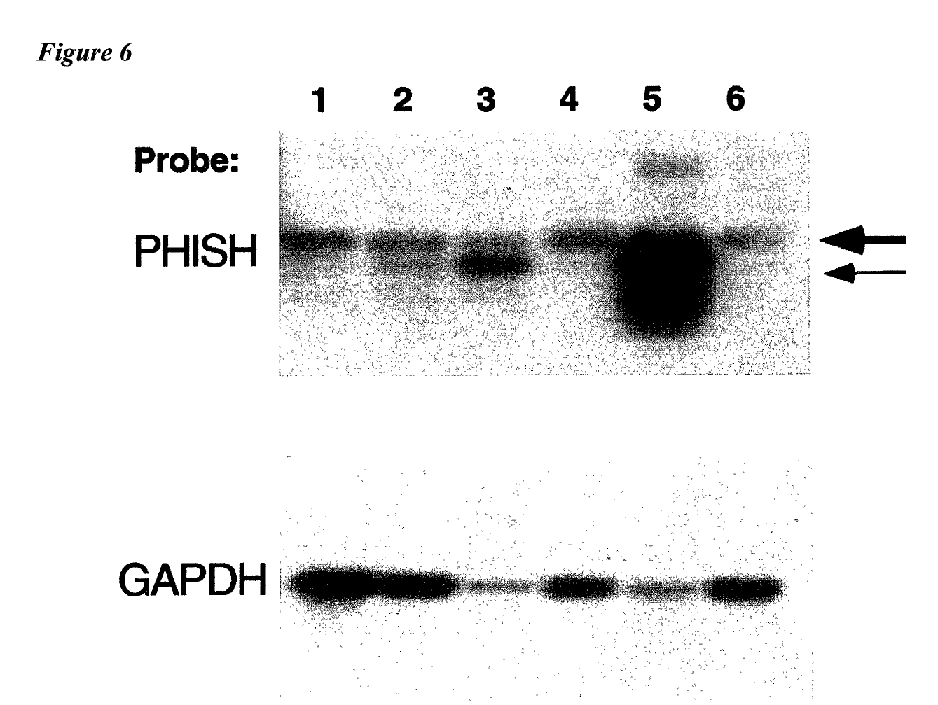

In order to determine the tissue distribution of PHISH we performed Northern blots on total RNA from several murine tissues, spleen, brain, heart, lung, thymus, and lymph using a probe derived from nucleotides 87 to 927, the putative coding region of the protein. Two RNA species that are approximately 1.2 to 1.4 kb were detected. These could represent products of alternative splicing of the gene. The larger of the two transcripts was detected in all tissues, and the smaller of the two transcripts was highly expressed in spleen and at lower levels in both heart and lung tissue (FIG. 6). In vitro transcription of the cDNA for PHISH isolated from the expression library also produced two transcripts that were approximately 1.2 -1.4 kb in size. The major transcript is very close in size to the larger of the two transcripts present in all murine tissues tested, while the minor product is close in size to the smaller transcript present in heart, lung, and spleen. This data suggests that the gene fragment isolated from the expression cloning library encodes the complete sequence of the gene.

Upon searching the human EST database with the nucleotide sequence of PHISH we found that PHISH had an 87% nucleotide sequence identity with that of a human EST derived from stem cells (locus AF150266). This EST encodes a cDNA that is 1.4 kb long and contains entire coding region of PHISH. The sequences of PHISH and the human EST differ significantly outside of the protein coding sequence. Moreover, three codons upstream from the putative start codon in the human EST are in frame stop codons. The high degree of sequence homology between PHISH and the human EST implies that they encode species-specific homologues of the same protein and that the gene for PHISH isolated from the expression screen encodes the entire protein.

Since PHISH contains only a PH domain and an SH2 domain, it is possible that PHISH functions as an adaptor protein. The PH domain could dock PHISH to 3′PPI generated at membrane receptor complexes and the SH2 domain could recruit phosphotyrosine containing protein(s) to such complexes. It is interesting to note that Skolnik and coworkers have isolated the PH domain of EST684797 from a homology search of human ESTs for PH domains that would be predicted to bind tightly to 3′PPIs. They have also shown that the PH domain of EST684797, which is highly homologous to the PH domain of PHISH binds tightly and selectively to PtdIns-3,4-P₂ and PtdIns-3,4,5-P₃ but not to PtdIns-4,5-P₂.

These studies demonstrate that coupled in vitro transcription/translation libraries can be used in conjunction with affinity isolation technology using synthetic phosphoinositides to isolate 3′PPI binding domains. Recently, several other methods have been described to isolate and clone 3′PPI binding proteins. One example is the use of resins coupled to phosphorylated inositol phosphates such as IP₃, or inositol phosphates linked to a glycerol moiety, to extract proteins from tissue extracts. Using these techniques PIP3BP and p42^{IP4}were extracted from total brain extracts. However, proteins expressed in low abundance are difficult to isolate by this procedure and furthermore, cDNA cloning requires multiple steps following affinity isolation.

Expression cloning of genes from cDNA libraries can circumvent some of the limitations of protein affinity isolation techniques. Two expression cloning techniques have been recently developed to identify 3′PPI binding proteins. The first involved the screening of a λgt 11 library with a crude mixture of brain phospholipids that had been phosphorylated in vitro by PI 3′-K. Screening of two murine derived cDNA libraries (from brain and adipocytes) yielded only one protein that bound tightly to PtdIns-3,4,5-P₃. This protein, called Grp1 (General Receptor for Phosphoinositides), contained a PH domain and an ARF-GEF domain that catalyzes 3′PPI dependent nucleotide exchange on mammalian ARFs. However, no other 3′PPI dependent binding protein was isolated in the screen possibly due to improper folding of the proteins.

Another recent expression cloning strategy for isolating 3′PPI binding proteins is the use of a modified yeast two-hybrid system. In this system, genes from a mammalian cDNA library were fused to a constitutively active Ras. The fusion proteins were then tested for their ability to rescue a temperature sensitive phenotype, due to a defect upstream of Ras, when coexpressed with constitutively active PI 3′-K. This system worked well in experiments where constitutively active Ras was fused to PH domains already known to bind to 3′PPIs. However, when tested with an expression library in yeast only Aktγ was isolated.

The screen described here is the first to isolate multiple 3′PPI binding targets from an expression library, including a novel protein. The basis for the better efficiency of this screen may stem from the fact that in vitro translated proteins are more likely to be properly folded. In addition, many internal initiation sites for translation allow for the independent and unoccluded expression of PH and other 3′PPI binding domains. We also used analogs of PtdIns-3,4-P₂ and PtdIns-3,4,5-P₃ that are synthetically pure and, as a result of modification in a distal region of the acyl chain, closely resemble the phosphorylated phosphatidylinositols that occur naturally. It has been shown that although the main binding of the protein is to the inositol head group, the contribution of the glycerol chain and the fatty acid side chains of the phosphatidylinositol are essential for specificity and tight binding to the protein. Our screening technique is not without drawbacks. For example, there can be non-specific binding of proteins to the avidin-coated beads and this non-specific binding may obscure the binding of other 3′PPI binding proteins with the same electrophoretic mobility. In addition, we isolated only C-terminal PH domains, possibly because our library was made using oligo-dT to prime cDNA synthesis from the 3′ poly-A tail of mRNAs. In addition, our screen thus far has isolated only strong binders of 3′PPIs indicating that our binding conditions may be too stringent to accommodate weaker binding proteins.

In summary, we have described a novel and effective way for isolating and cloning 3′PPI binding proteins from expression libraries. The technique described here has broad applications for the isolation of binding partners for other phosphoinositide polyphosphates or other lipid products.

1. A method for identifying a cellular component which binds to a lipid moiety comprising:
a. providing a lipid bait moiety being derivatized to a solid support;
b. contacting the lipid bait moiety with a library of cellular components;
c. identifying those members of the cellular component library which specifically bind to the lipid bait moiety.

2. The method of claim 1, wherein the lipid bait moiety is a phospholipid.

3. The method of claim 2, wherein the lipid bait moiety is a phospholipid selected from the group consisting of phosphatidylethanolamines, phosphatidylcholines, phosphatidylserines, phosphatidylglycerols, phosphatidylinositols, polyphosphatidylinositols, and diphosphatidylglycerols.

4. The method of claim 2, wherein the phospholipid is derivatized to the solid support through a cross-linking moiety which is covalently attached to a phosphate head group of the phospholipid.

5. The method of claim 1, wherein the lipid bait moiety a plasmalogen or a sphingolipid.

6. The method of claim 1, wherein the library of cellular components is a polypeptide library.

7. The method of claim 6, wherein the polypeptide library is an expression library.

8. The method of claim 7, wherein the polypeptide library is derived from replicable genetic display packages.

9. The method of claim 6, wherein the protein library is a cell lysate or partially purified protein preparation.

10. The method of claim 1, 6 or 9, wherein the identity of those members of the cellular component library which specifically bind to the lipid bait moiety is determined by mass spectroscopy

11. A drug screening assay comprising:
a. providing a reaction mixture including a cellular component identified in claim 1 as able to specifically bind to the lipid bait moiety;
b. contacting the cellular component with a test compound;
c. determining if the test compound binds to the cellular component.

12. The method of claim 11, wherein the test compound which is identified as able to bind to the cellular component is further tested for the ability to inhibit or mimic the activity of a lipid moiety.

13. The method of claim 11, wherein the reaction mixture is a whole cell.

14. The method of claim 11, wherein the reaction mixture is a cell lysate or purified protein composition.

15. A method of conducting a drug discovery business comprising:
a. providing a lipid bait moiety being derivatized to a solid support;
b. contacting the lipid bait moiety with a library of cellular components;
c. identifying those members of the cellular component library which specifically bind to the lipid bait moiety.
providing a reaction mixture including a cellular component identified in step (c) as able to specifically bind to the lipid bait moiety;
e. contacting the cellular component with a test compound;
f. determining if the test compound binds to the cellular component;
g. further testing those test compound identified in step (f) as able to bind to the cellular component for the ability to inhibit or mimic the activity of a lipid moiety; and
h. formulating a pharmaceutical preparation including one or more compounds identified in step (g) as able to inhibit or mimic the activity of a lipid moiety.

16. A method of conducting a target discovery business comprising:
a. providing a lipid bait moiety being derivatized to a solid support;
b. contacting the lipid bait moiety with a library of cellular components;
c. identifying those members of the cellular component library which specifically bind to the lipid bait moiety.
d. licensing, to a third party, the rights for drug development for a cellular component identified in step (c) as able to specifically bind to the lipid bait moiety.
